(19) United States
(12) Patent Application Publication
(10) Pub. No.: US 2024/0068971 A1
(43) Pub. Date: Feb. 29, 2024

(54) MOLECULARLY IMPRINTED POLYMER SENSOR FOR PER- AND POLY-FLUOROALKYL SUBSTANCES (PFAS)

(71) Applicant: The MITRE Corporation, McLean, VA (US)

(72) Inventors: Jamie Amanda GROVE, Fairfax, VA (US); Kathy T. HUYNH, Falls Church, VA (US); Emiko ZUMBRO, Arlington, VA (US); Joseph John Patrick ROBERTS, Clifton, VA (US); Sean Mitchell OLIVER, McLean, VA (US); Lindsey Jordan GOODNIGHT, McLean, VA (US); Nathan Isaac WALTON, Potomac, MD (US)

(73) Assignee: The MITRE Corporation, McLean, VA (US)

(21) Appl. No.: 18/456,411

(22) Filed: Aug. 25, 2023

Related U.S. Application Data
(60) Provisional application No. 63/401,378, filed on Aug. 26, 2022.

(51) Int. Cl. G01N 27/12; G01N 33/00

(52) U.S. Cl. CPC G01N 27/126; G01N 33/0047

(57) ABSTRACT

A method for quantifying amounts of per- and poly-fluoroalkyl substances (PFAS) may use a PFAS detection system comprising a working electrode and one or more processors. The working electrode may have a polymer layer disposed on its surface that comprises a plurality of affinity sites for detecting a plurality of PFAS molecules. Each of the plurality affinity sites may have been created using a template PFAS. The method may comprise detecting, at the working electrode, a plurality PFAS molecules that have bonded to one or more of the plurality of affinity sites; and determining, by the one or more processors and based on the detected plurality of PFAS molecules that are bonded to the one or more affinity sites, a concentration of PFAS in an environment.

### CROSS-REFERENCE TO RELATED APPLICATIONS

This application claims the benefit of U.S. Provisional Application No. 63/401,378, filed Aug. 26, 2022, the entire contents of which is incorporated herein by reference.

### FIELD OF THE DISCLOSURE

The present disclosure relates generally to systems, methods, and sensors for measuring concentrations of per- and poly-fluoroalkyl substances (PFAS) in an environment (e.g., a body of water) as well as methods of manufacturing PFAS sensors.

### BACKGROUND OF THE DISCLOSURE

Per- and poly-fluoroalkyl substances (PFAS) are a class of over 4700 synthetic chemical compounds that are widely used in the manufacturing of products such as cookware, firefighting foams, textiles, water repellants, and stain repellants. In recent years, PFAS have been identified as potentially hazardous pollutants in the environment. PFAS exposure has been linked to a number of human and environmental health issues. Even though many of the large number of PFAS are believed to be dangerous, conventional real-time, in situ PFAS sensing systems may be configured to detect only a single type of PFAS among thousands. Some recently developed PFAS sensing systems may collapse or degrade when exposed to liquids, making them prone to false positive results and ill-suited to in situ detection of PFAS in aqueous environments. Accordingly, efficient, cost-effective, robust, and highly sensitive PFAS sensing systems are needed to effectively detect and quantify amounts of PFAS in contaminated environments.

### SUMMARY OF THE DISCLOSURE

The mechanisms through which PFAS are transported through the environment are the subject of ongoing research. Efficient monitoring of the concentrations of PFAS is essential to determining and mitigating the effects of PFAS exposure in humans. Conventional in situ sensing systems are highly specialized; generally, conventional systems configured to be used in the field may only be capable of detecting one of the thousands of types of PFAS. Other methods (e.g., mass spectrometry) that are capable of detecting multiple PFAS at once may not be amenable to in-field measurements (e.g., may require environmental samples to be brought back to a laboratory for testing) and may require operators to have high levels of expertise or training.

The present disclosure provides systems, methods, and sensors for simultaneous, in situ detection and quantification of PFAS using a single sensor, as well as methods for manufacturing a PFAS sensor. In one or more examples, PFAS measurements may be executed in real time at any location of interest. The PFAS sensor may comprise a plurality of electrodes that have been modified with molecularly imprinted polymers (MIPs). The MIPs may have a low selectivity between PFAS of a particular class but high selectivity against other, non-PFAS co-contaminants. In one or more examples, this may allow a single modified electrode to respond to multiple different PFAS at once. Multiplexing multiple modified electrodes together may thus create a single system capable of simultaneously detecting and quantifying amounts of over seventy types of PFAS. In one or more examples, a PFAS sensor is capable of detecting and quantifying over ten types of PFAS, over twenty types of PFAS, over 50 types of PFAS, over 70 types of PFAS, or over 100 types of PFAS.

In other examples, the MIPs are specific to a particular PFAS, such that the sensor is capable of selectively measuring the quantity of a specific PFAS in the environment. The methods of manufacturing described herein may allow a user to create PFAS sensors with MIPs having affinity sites exhibiting a desired binding behavior with PFAS, such as specific or non-specific binding to various PFAS targeted by a detection and quantification method. Improved manufacturing methods may also result in MIPs with higher mechanical stability in aqueous environments and improved binding behavior with target PFAS. Accordingly, the PFAS sensors described herein may be more robust and resilient than conventional PFAS sensors, enabling users to detect PFAS in situ after incubation in aqueous environments where PFAS may be found.

In one or more examples, a method for quantifying amounts of per- and poly-fluoroalkyl substances (PFAS) using a PFAS detection system comprising a working electrode, a polymer layer disposed on a surface of the working electrode and comprising a plurality of affinity sites for detecting a plurality of PFAS molecules having different molecular structures, wherein the each of the plurality affinity sites were created using a template PFAS and one or more processors comprises: detecting, at the working electrode, a plurality of PFAS molecules having different molecular structures that have bonded to one or more of the plurality of affinity sites; and determining, by the one or more processors and based on the detected plurality of PFAS molecules that are bonded to the one or more affinity sites, a concentration of PFAS in an environment.

In one or more examples of the method, the plurality of affinity sites comprise multiple affinity sites that were created using the same template PFAS.

In one or more examples of the method, the multiple affinity sites that were created using the same template PFAS are configured to bind to one or more PFAS having molecular structures that are different from a molecular structure of the template PFAS.

In one or more examples of the method, the one or more PFAS have molecular structures that are at least 60% identical to the molecular structure of the template PFAS.

In one or more examples of the method, the multiple affinity sites that were created using the same template PFAS are configured to bind to at least two PFAS having molecular structures that are different from the molecular structure of the template PFAS.

In one or more examples of the method, the determined concentration of PFAS in the environment is a total concentration of all PFAS having different molecular structures that can bind to the one or more affinity sites.

In one or more examples of the method, detecting the plurality of PFAS molecules that are bonded to one or more of the plurality of affinity sites comprises: generating, using a voltage source, a varying potential difference between the working electrode and a reference electrode; and detecting, as the potential difference is varied, a current response of the working electrode.

In one or more examples of the method, a potentiostat is used to generate the potential difference and to detect the current response.

In one or more examples of the method, determining the concentration of PFAS in the environment comprises: determining a difference between the detected current response of the working electrode to a current response of the working electrode in an uncontaminated environment; and computing the concentration of PFAS based on the determined difference between the detected current response and the current response in the uncontaminated environment.

In one or more examples of the method, the method comprises, prior to detecting the plurality PFAS molecules: incubating the working electrode and the polymer layer in an aqueous environment containing PFAS molecules to allow one or more PFAS molecules to bind to one or more of the affinity sites; and exposing the working electrode and the polymer layer to a sensing solution, wherein the detecting of the plurality of PFAS molecules occurs in the sensing solution.

In one or more examples of the method, the method comprises, prior to detecting the plurality PFAS molecules, incubating the working electrode and the polymer layer in an electrolyte solution.

In one or more examples of the method, the polymer layer is formed via molecular imprinting.

In one or more examples, a system for quantifying amounts of per- and poly-fluoroalkyl substances (PFAS) comprises: a working electrode; a polymer layer disposed on a surface of the working electrode and comprising a plurality of affinity sites for detecting a plurality of PFAS molecules having different molecular structures, wherein each of the plurality affinity sites were created using a template PFAS; and one or more processors; wherein the working electrode is configured to detect a plurality of PFAS molecules having different molecular structures that have bonded to one or more of the plurality of affinity sites; and wherein the one or more processors are configured to determine, based on the detected plurality of PFAS molecules that are bonded to the one or more affinity sites, a concentration of PFAS in an environment.

In one or more examples of the system, the plurality of affinity sites comprise multiple affinity sites that were created using the same template PFAS.

In one or more examples of the system, the multiple affinity sites that were created using the same template PFAS are configured to bind to one or more PFAS having molecular structures that are different from a molecular structure of the template PFAS.

In one or more examples of the system, the one or more PFAS have molecular structures that are at least 60% identical to the molecular structure of the template PFAS.

In one or more examples of the system, the multiple affinity sites that were created using the same template PFAS are configured to bind to at least two different PFAS having molecular structures that are different from the molecular structure of the template PFAS.

In one or more examples of the system, the determined concentration of PFAS in the environment is a total concentration of all PFAS having different molecular structures that can bind to the one or more affinity sites.

In one or more examples of the system, detecting the plurality of PFAS molecules that are bonded to one or more of the plurality of affinity sites comprises: generating a varying potential difference between the working electrode and a reference electrode; and detecting, as the potential difference is varied, a current response of the working electrode.

In one or more examples of the system, a potentiostat is used to generate the potential difference and to detect the current response.

In one or more examples of the system, determining the concentration of PFAS in the environment comprises: determining a difference between the detected current response of the working electrode to a current response of the working electrode in an uncontaminated environment; and computing the concentration of PFAS based on the determined difference between the detected current response and the current response in the uncontaminated environment.

In one or more examples of the system, the polymer layer is formed via molecular imprinting.

In one or more examples of the system, the working electrode is operable to detect the plurality of PFAS molecules after exposing the polymer layer and the working electrode to an aqueous sample environment for at least 30 mins.

In one or more examples, a non-transitory computer readable storage medium stores instructions configured to be executed by a PFAS detection system comprising: a working electrode; a polymer layer disposed on a surface of the working electrode and comprising a plurality of affinity sites for detecting a plurality of PFAS molecules having different molecular structures, wherein each of the plurality affinity sites were created using a template PFAS; and one or more processors, wherein, when executed by the one or more processors, the instructions are configured to cause the PFAS detection system to: detect, at the working electrode, a plurality PFAS molecules having different molecular structures that have bonded to one or more of the plurality of affinity sites; and determine, by the one or more processors and based on the detected plurality of PFAS molecules that are bonded to the one or more affinity sites, a concentration of PFAS in an environment.

In one or more examples of the non-transitory computer readable storage medium, the plurality of affinity sites comprise multiple affinity sites that were created using the same template PFAS.

In one or more examples of the non-transitory computer readable storage medium, the multiple affinity sites that were created using the same template PFAS are configured to bind to one or more PFAS having molecular structures that are different from a molecular structure of the template PFAS.

In one or more examples of the non-transitory computer readable storage medium, the one or more PFAS have molecular structures that are at least 60% identical to the molecular structure of the template PFAS.

In one or more examples of the non-transitory computer readable storage medium, the multiple affinity sites that were created using the same template PFAS are configured to bind to at least two PFAS having molecular structures that are different from the molecular structure of the template PFAS.

In one or more examples of the non-transitory computer readable storage medium, the determined concentration of PFAS in the environment is a total concentration of all PFAS having different molecular structures that can bind to the one or more affinity sites.

In one or more examples of the non-transitory computer readable storage medium, wherein detecting the plurality of PFAS molecules that are bonded to one or more of the plurality of affinity sites comprises: generating, using a voltage source, a varying potential difference between the working electrode and a reference electrode; and detecting, as the potential difference is varied, a current response of the working electrode.

In one or more examples of the non-transitory computer readable storage medium, a potentiostat is used to generate the potential difference and to detect the current response.

In one or more examples of the non-transitory computer readable storage medium, determining the concentration of PFAS in the environment comprises: determining a difference between the detected current response of the working electrode to a current response of the working electrode in an uncontaminated environment; and computing the concentration of PFAS based on the determined difference between the detected current response and the current response in the uncontaminated environment.

In one or more examples of the non-transitory computer readable storage medium, the polymer layer is formed via molecular imprinting.

In one or more examples, a method of manufacturing a PFAS sensor is provided, the method comprising: forming a polymer layer on a working electrode comprising: contacting the working electrode with a solution comprising a plurality of monomers and template PFAS, and an acid, and electropolymerizing the monomers to form the polymer layer and trap the template PFAS in the polymer layer; extracting the template PFAS from the polymer layer to create a plurality of affinity sites in the polymer layer; and incubating the working electrode and the polymer layer in an electrolyte solution.

In one or more examples of the method, the acid is HCl.

In one or more examples of the method, extracting the plurality of template PFAS comprises washing the polymer layer with an acetone solution.

In one or more examples of the method, the acetone solution is a 1:1 acetone:water solution.

In one or more examples of the method, the plurality of affinity sites are configured to bind to one or more PFAS having molecular structures that are different from a molecular structure of the template PFAS.

In one or more examples of the method, the acetone solution is a 1:1: acetone:acid solution.

In one or more examples of the method, the plurality of affinity sites are configured to bind to one or more PFAS having molecular structures that are the same as a molecular structure of the template PFAS.

In one or more examples of the method, the electrolyte solution is an ammonia solution.

In one or more examples of the method, the working electrode and the polymer layer are incubated in the electrolyte solution for at least 12 hours.

In one or more examples, a PFAS sensor for quantifying amounts of per- and poly-fluoroalkyl substances (PFAS) is provided, the sensor comprising: a working electrode; and a polymer layer disposed on the working electrode and comprising a plurality of affinity sites for binding a plurality of PFAS molecules, wherein each of the plurality of affinity sites were created using a template PFAS; and wherein the working electrode is operable to detect PFAS molecules bound to the affinity sites after exposing the working electrode and the polymer layer to an aqueous sample environment for at least 30 minutes.

In one or more examples of the sensor, the polymer layer is stable in the aqueous sample environment for at least 2 hours.

In one or more examples of the sensor, the working electrode is operable to detect the PFAS molecules after exposing the working electrode and the polymer layer to the aqueous sample environment for at least 12 hours.

In one or more examples of the sensor, the plurality of affinity sites are configured to bind to PFAS molecules having molecular structures that are different from a molecular structure of the template PFAS.

In one or more examples of the sensor, the PFAS molecules have molecular structures that are at least 60% identical to the molecular structure of the template PFAS.

In one or more examples of the sensor, the plurality of affinity sites are configured to bind to at least two PFAS molecules having molecular structures that are different from the molecular structure of the template PFAS.

In one or more examples of the sensor, the plurality of affinity sites are configured to bind to PFAS molecules having molecular structures that are the same as a molecular structure of the template PFAS.

### BRIEF DESCRIPTION OF THE FIGURES

The invention will now be described, by way of example only, with reference to the accompanying drawings.

FIGS. 1A-1B illustrate two exemplary PFAS types.

FIG. 2 illustrates an exemplary system for quantifying amounts of PFAS in an environment, according to one or more examples of the present disclosure.

FIG. 3 illustrates an exemplary method for quantifying amounts of PFAS in an environment, according to one or more examples of the present disclosure.

FIG. 4A illustrates exemplary binding behavior of PFAS affinity sites, according to one or more examples of the present disclosure.

FIG. 4B illustrates an exemplary template PFAS and exemplary PFAS that an electrode modified with the template PFAS may be capable of detecting.

FIG. 4C illustrates an exemplary template PFAS and exemplary PFAS that an electrode modified with the template PFAS may be capable of detecting.

FIG. 5 illustrates an exemplary method for modifying an electrode with PFAS affinity sites according to one or more examples of the present disclosure.

FIG. 6 illustrates exemplary responses in an uncontaminated and in a contaminated environment of an electrode that is modified with PFAS affinity sites, according to one or more examples of the present disclosure.

FIG. 7 illustrates an exemplary three-electrode system for measuring a current response of a modified electrode, according to one or more examples of the present disclosure.

FIG. 8 illustrates an exemplary potentiostat for controlling a potential difference between a reference electrode and a working electrode, according to one or more examples of the present disclosure.

FIG. 9 illustrates an exemplary method for determining a concentration of PFAS using an electrode modified with PFAS affinity sites, according to one or more examples of the present disclosure.

FIG. 10 illustrates an exemplary system for quantifying amounts of multiple classes of PFAS associated with multiple modified electrodes, according to one or more examples of the present disclosure.

FIG. 11 illustrates exemplary scan parameters for characterization of an electrode via cyclic voltammetry (CV) and differential pulse voltammetry (DPV), according to one or more examples of the present disclosure.

FIG. 12 exemplary scan patemeters for MIP fabrication, according to one or more examples of the present disclosure.

FIGS. 13A-13B illustrate the experimental results from two trials conducted on prior art PFAS sensors, according to one or more examples of the present disclosure.

FIG. 14A illustrates experimental data collected from an exemplary PFAS sensor manufactured by prior art manufacturing methods when the sensor is incubated in a water-only experimental solution, according to one or more examples of the present disclosure.

FIG. 14B illustrates the results of experimental trails conducted on a PFAS sensor fabricated using an acetone solution for electropolymerization of the polymer layer, according to one or more examples of the present disclosure.

FIGS. 15A-15B illustrate the results of experimental trails conducted by incubating PFAS sensors in water alone, according to one or more examples of the present disclosure.

FIGS. 16A-16B illustrate the results of experimental trails conducted on PFAS sensors fabricated using an acetone solution for electropolymerization of the polymer layer, conducted by incubating the PFAS sensors in water alone and then spiking the water with various amounts of PFOS and PFOA, according to one or more examples of the present disclosure.

FIGS. 17A-17B illustrate the results of experimental trials conducted on non-specific PFAS sensors, following fabrication of the PFAS sensors using an acetone and acid solution for electropolymerization of the polymer layer and a mixture of acetone and water for template extraction. Results are shown after incubation in experimental test solutions containing various concentrations of PFOS and PFOA for 40 minutes, according to one or more examples of the present disclosure.

FIGS. 18A-18D illustrate the results of experimental trails conducted on PFAS sensors manufactured to detect specific PFAS species, the sensors fabricated using an acetone and acid solution for electropolymerization of the polymer layer and a mixture of acetone and acid for template extraction. Results shown after incubation in experimental test solutions containing various amounts of the PFAS species for 40 and 60 minutes, according to one or more examples of the present disclosure.

### DETAILED DESCRIPTION OF THE DISCLOSURE

Per- and poly-fluoroalkyl substances (PFAS) are synthetic organic chemical compounds comprising at least one methyl or methylene carbon atom that is fully fluorinated. Due primarily to their effectiveness as surfactants, PFAS have seen widespread use in a large number of products, including non-stick cookware, firefighting foams, water repellant clothing, and adhesives. Despite their usefulness, there has been growing concern over the potential adverse effects of PFAS exposure on human health. PFAS do not break down quickly when left in the environment (e.g., as industrial waste). As a result, PFAS can accumulate in soils and in bodies of water that are used by humans, thereby increasing the likelihood that said humans are exposed.

Quantifying PFAS levels in environments that humans frequently interact with (e.g., bodies of water that serve as drinking water sources) is a necessary step in monitoring human exposure to PFAS. However, the large number of different PFAS has posed a significant challenge to efforts to study total environmental PFAS levels. Additionally, the mechanisms of PFAS transport through the environment are not currently well-understood. Even though many of the large number of PFAS are believed to be dangerous, conventional real-time, in situ PFAS sensing systems may be configured to detect only a single type of PFAS among thousands. Determining concentrations of many PFAS at a single location using conventional methods may be inefficient and cost-ineffective because a large number of different sensors may be required.

The present disclosure provides systems, methods, and sensors for quantifying amounts of multiple different PFAS using a single sensor system. The present disclosure also provides methods for manufacturing PFAS sensors. In one or more examples, the sensor may include a working electrode that has been modified with a plurality of affinity sites for detecting one or more PFAS. When the sensor is placed in an environment that is contaminated with PFAS, a plurality of PFAS molecules may bind to one or more of the affinity sites. The binding of the PFAS molecules to the affinity sites may have a measurable impact on the behavior of the electrode. This measurable impact can then be related to the concentration of specific PFAS molecules or overall PFAS concentration in the contaminated environment.

Each of the plurality of affinity sites that modify the working electrode may be created using a template PFAS molecule. The plurality of affinity sites may be configured to select for a particular molecular structure that is present in the template PFAS molecule. As such, when exposed to a contaminated environment, the affinity sites on the modified working electrode may be configured to bind to one or more PFAS having a molecular structure that is identical to the template PFAS molecule (e.g., a PFAS that is identical to the template PFAS).

There may also exist one or more PFAS which, while not being identical to the template PFAS, comprise similar molecular structures to the molecular structure present in the template PFAS. In one or more examples, the selectivity of the affinity sites to be exploited to cause the similar PFAS to bind to the working electrode in addition to PFAS that are identical to the template molecule. That is, in one or more examples, a working electrode that is modified with affinity sites that are created using a single template PFAS may be capable of responding to multiple PFAS in addition to the template PFAS. A single working electrode may therefore be capable of sensing the presence of two or more PFAS simultaneously.

Quantifying amounts of PFAS using a single sensor as described above may involve detecting a plurality of PFAS that have bonded to the affinity sites and then determining a concentration of PFAS based on the detected plurality of PFAS. In one or more examples, the determined concentration of PFAS may include a total concentration of all PFAS having different molecular structures that can bind to the plurality of affinity sites. For instance, if the affinity sites can bind to any of four PFAS species, the determined concentration of PFAS may include a total concentration of all four PFAS species. However, in other examples, the determined concentration of PFAS may include a concentration of a single PFAS species or a particular subset of PFAS species that can bind to the affinity sites.

The systems, methods, and sensors of the present disclosure may allow for the detection and quantification of low concentrations of PFAS. In some examples, the systems, methods, and sensors may allow for the simultaneous detection and quantification of multiple different PFAS with a single sensor. Since concentrations of multiple PFAS can be comprehensively quantified with a single sensor, the systems, methods, and sensors described herein may provide an efficient and cost-effective alternative to conventional PFAS sensing technologies. The methods described herein may also enable the manufacture of PFAS sensors having a desired binding affinity, such as specific or non-specific binding affinity with one or more PFAS, as well as improved stability in aqueous sample environments. Advantageously, the systems, methods, and sensors of the present disclosure may, in one or more examples, be used and/or executed at the site of the contaminated environment (e.g., an aqueous environment), allowing PFAS measurements to be carried out in situ without need for specialized laboratory equipment or training.

Reference will now be made in detail to implementations and embodiments of various aspects and variations of systems, methods, and sensors described herein. Although several exemplary variations of the systems, methods, and sensor are described herein, other variations of the systems, methods, and sensors may include aspects of the systems, methods, and sensors described herein combined in any suitable manner having combinations of all or some of the aspects described.

In the following description of the various embodiments, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure, in one or more examples, also relates to a device for performing the operations herein. This device may be specially constructed for the required purposes, or it may comprise a general purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each connected to a computer system bus. Furthermore, the computing systems referred to in the specification may include a single processor or may be architectures employing multiple processor designs, such as for performing different functions or for increased computing capability. Suitable processors include central processing units (CPUs), graphical processing units (GPUs), field programmable gate arrays (FPGAs), and ASICs.

### Examples of PFAS

As discussed above, PFAS are synthetic organic chemical compounds comprising at least one methyl or methylene carbon atom that is fully fluorinated (i.e., at least one methyl/methylene carbon atom wherein all bonded hydrogen has been replaced with fluorine). PFAS are used in large number of common products, including non-stick cookware, water repellant clothing, and stain repellants. Quantifying amounts of PFAS in the environment is essential to determining whether—and to what extent—people have been exposed to these potentially toxic chemicals as well as determining appropriate remediation methods.

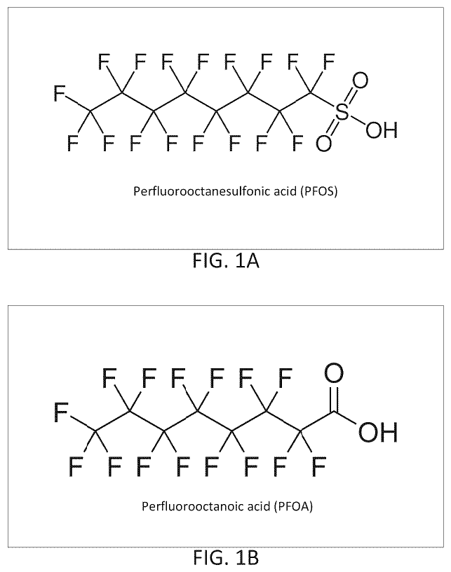

FIGS. 1A-1B illustrate two exemplary subgroups of PFAS. As shown, each PFAS contains at least one fully fluorinated methyl or methylene carbon atom. FIG. 1A illustrates perfluorooctanesulfonic acid (i.e., PFOS), a type of perfluorosulfonic acid. PFOS, which is believed to be linked to adverse reproductive, developmental, and immunological effects in humans, was, until the early 2000s, a key ingredient in many stain repellants manufactured in the United States (e.g., 3M's Scotchgard), and is currently produced in countries outside of the US (e.g., China). FIG. 1B illustrates perfluorooctanoic acid (i.e., PFOA), a type of perfluorocarboxylic acid. PFOA is widely used in carpeting, textiles, and sealants, and has been detected in the blood of over 98% of the US population. PFOA has been linked with multiple cancers and other diseases, including kidney cancer.

The compounds shown in FIGS. 1A-1B are only two examples of the over 4700 types of PFAS. Since PFAS display significant in-group variation in structure and chemical/physical properties, PFAS monitoring systems and sensors are typically configured to detect only a single type of PFAS. As such, gathering comprehensive data on the amount of PFAS at a given location can be extremely inefficient. Furthermore, many PFAS monitoring systems require environmental samples to be removed from the testing location, treated with various chemicals, and measured using complex, expensive equipment in a laboratory. These processes are often time consuming and costly, as they cannot be performed in situ and may require a high level of expertise to be properly executed.

### Systems and Methods for Detecting and Quantifying PFAS

In order to effectively monitor human exposure to PFAS and to assess health issues related to PFAS pollution, the levels of PFAS in the environment must be measured. The systems and methods described herein can utilize a single sensor to determine concentrations of one or more PFAS. In one or more examples, the sensor may be configured to determine said PFAS concentrations in situ, i.e., at the testing location.

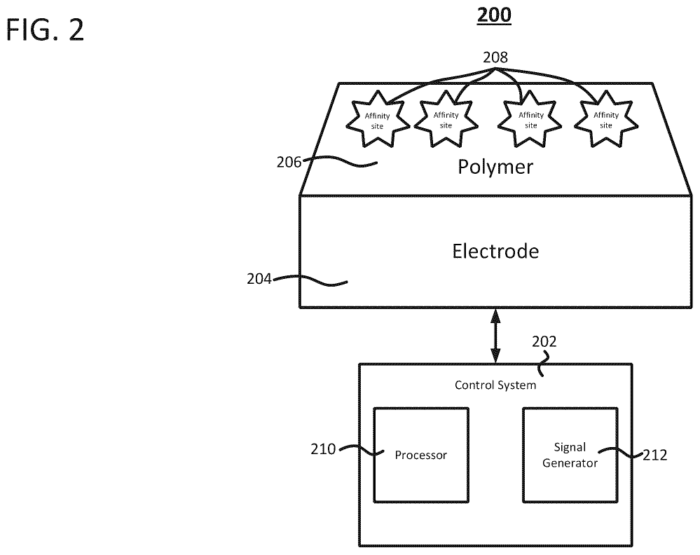

FIG. 2 illustrates an exemplary system for quantifying amounts of PFAS in an environment, according to one or more examples of the present disclosure. Specifically, FIG. 2 shows a system 200 comprising a control system 202 that is coupled to an electrode 204. In one or more examples, electrode 204 may be modified with a polymer layer 206 comprising a plurality of affinity sites 208. The plurality of affinity sites 208 may have been created using a template PFAS and, in one or more examples, may be configured to bind to one or more PFAS having an identical or nearly identical structure to the template PFAS.

In one or more examples, control system 202 may comprise a processor 210 and a signal generator 212. Signal generator 212 may be electrically coupled to electrode 204 and may be configured to apply an electrical signal to electrode 204. In one or more examples, when an electrical signal is applied to electrode 204, a charge-transferring reaction (e.g., an oxidation-reduction reaction) may be induced between electrode 204 and one or more atoms in the surrounding environment. Processor 210 may be configured to receive data related to an electrical signal applied to electrode 204 and a subsequently induced charge-transferring reaction.

In one or more examples, one or more physical properties of electrode 204 may change as a function of the amount of PFAS that bind to the plurality of affinity sites 208. For instance, one or more examples, the surface capacitance of electrode 204 may change as a function of the amount of PFAS that bind to the plurality of affinity sites 208. In one or more examples, changes in one or more physical properties of electrode 204 may be monitored and used to determine a concentration of PFAS in an environment.

Electrode 204 may be a cathode or an anode. Electrode 204 may be configured to react with one or more elements found in water when an electrical signal is applied by signal generator 212. This may allow system 200 to operate directly (i.e., conduct PFAS measurements) in environments that are likely to be polluted with PFAS such as rivers, lakes, ponds, or other bodies of water. In one or more examples, an oxygen reduction reaction may occur at surfaces of electrode 204 that are exposed to the surrounding environment when an electrical signal is applied to electrode 204.

In one or more examples, electrode 204 may comprise one or more conducting or semi-conducting materials. In one or more examples, electrode 204 may comprise gold. In one or more examples, electrode 204 may comprise glassy carbon. In one or more examples, electrode 204 may be a macro-electrode, a micro-electrode, or a screen-printed electrode. In one or more examples, electrode 204 may be greater than or equal to 0.01, 0.1, 0.5, 1, 10, 10², 10³, 10⁴, or 10⁵ micrometers in diameter. In one or more examples, electrode 204 may be less than or equal to 0.01, 0.1, 0.5, 1, 10, 10², 10³, 10⁴, or 10⁵ micrometers in diameter. In one or more examples, electrode 204 may be between about 0.01-0.1, 0.01-0.5, 1-10², 1-10³, 1-10⁴, or 1-10⁵ micrometers in diameter.

In one or more examples, polymer layer 206 may comprise an electro-polymerizable polymer. In one or more examples, polymer layer 206 may comprise poly(o-phenylenediamene). In one or more examples, polymer layer 206 may comprise polydopamine. In one or more examples, polymer layer 206 is formed by electropolymerizing monomers in a solution (e.g., an acetone or methanol solution) to cause the monomers to self-assemble into a polymer layer on the electrode 204. In one or more examples, the polymerization of the polymer layer 206 occurs in the presence of template PFAS molecules, such that one or more of the template PFAS become trapped in the polymer layer when the monomers self-assemble. Extraction of the template PFAS from the polymer layer 206 may form the affinity sites 208 in the polymer layer.

In one or more examples, the polymerization of the monomers occurs in a low-pH solution (e.g., a solution with a pH of less than 3 or less than 2.5). The low pH of the solution may result in the formation of a polymer layer 206 having increased mechanical stability compared with polymer layers formed in less acidic solutions. For instance, the polymer layer 206 may have increased backbone stiffness, cross-linking, covalent bonding, and/or non-covalent interactions that increase the mechanical stability of the polymer. Accordingly, the polymer layer 206 may be stable in aqueous environments for at least 2 minutes, at least 5 minutes, at least 10 minutes, at least 30 minutes, at least 1 hour, at least 2 hours, at least 6 hours, or at least 12 hours. In one or more examples, the sensor (e.g., the working electrode of the sensor) is operable to detect PFAS after exposing the sensor (e.g., the working electrode and/or polymer layer) to an aqueous sample environment for at least 2 minutes, at least 5 minutes, at least 10 minutes, at least 30 minutes, at least 1 hour, at least 2 hours, at least 6 hours, or at least 12 hours.

In one or more examples, polymer layer 206 may be greater than or equal to 5, 10, 20, 30, 40, or 50 nanometers thick. In one or more examples, polymer layer 206 may be less than or equal to 5, 10, 20, 30, 40, or 50 nanometers thick. In one or more examples, polymer layer 206 may be between about 1-10, 10-20, 20-30, 30-40, 40-50, or 50-100 nanometers thick.

In one or more examples, the plurality of affinity sites 208 may comprise multiple affinity sites that were created using the same template PFAS. The multiple affinity sites that were created using the same template PFAS may be configured to bind to one or more PFAS having molecular structures that are different from a molecular structure of the template PFAS. In one or more examples, the multiple affinity sites that were created using the same template PFAS may be configured to bind to all of the shorter chains of PFAS of the template molecule. In one or more examples, the multiple affinity sites that were created using the same template PFAS may be capable of binding to at least 2, at least 10, at least 20, at least 30, at least 40, at least 50, at least 60, at least 70, at least 80, at least 90, at least 100, or at least 1000 different PFAS having molecular structures that are different from the molecular structure of the template PFAS.

In one or more examples, system 200 may be used to quantify a concentration of PFAS in an environment by placing electrode 204 in the environment and determining, using processor 210, amounts of PFAS in the environment based on a response of electrode 204. In one or more examples, control system 202 may be configured to receive user inputs from a user. The user inputs may be configured to control signal generated by signal generator 212 in order to initiate a measurement of PFAS in the environment. In one or more examples, control system 202 may be configured to connect to a memory system and/or a user interface system in order to store or display data collected with system 200 and/or allow users to interact with or control system 200.

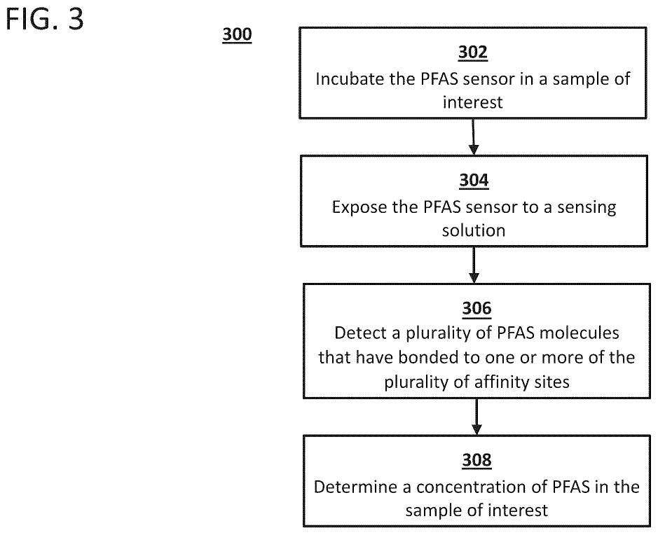

FIG. 3 illustrates an exemplary method for quantifying amounts of PFAS in an environment, according to one or more examples of the present disclosure. Specifically, FIG. 3 shows a method 300 for determining a concentration of PFAS in an environment, such as an aqueous environment containing PFAS. In one or more examples, method 300 may be executed by a system comprising a working electrode that is modified with a polymer layer including a plurality of PFAS affinity sites for binding to one or more PFAS in the environment (e.g., system 200 shown in FIG. 2). The working electrode and the polymer layer may be referred to as a “sensor” or “PFAS sensor”.

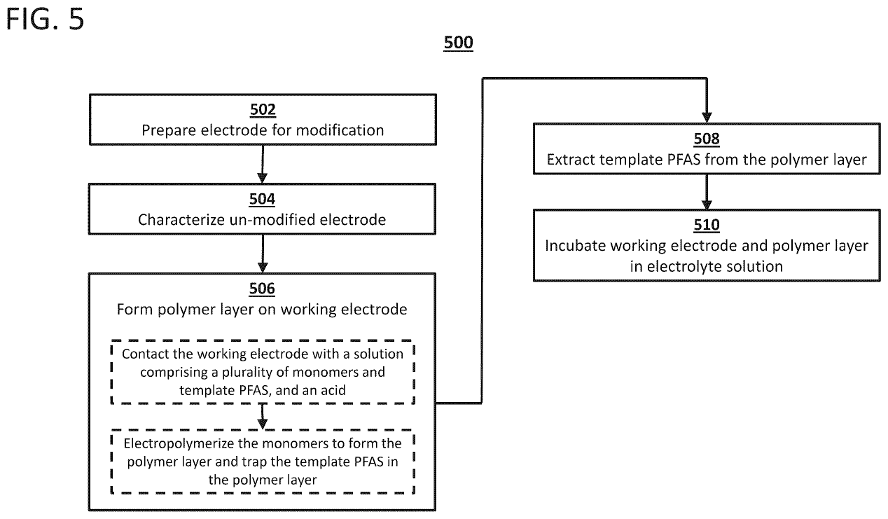

In one or more examples, the method 300 may comprise a step 302, wherein the PFAS sensor is incubated in a sample of interest, such as a fluid sample containing PFAS, to allow one or more PFAS to bind to one or more of the affinity sites. In one or more examples, the sample of interest is a fluid sample taken from the environment, such as a fluid sample from a river, a lake, surface water, or some other body of water. In one or more examples, the sensor is incubated in situ, for instance, by contacting the sensor with an environmental sample that has not been removed from the environment. In some examples, incubating the sensor includes incubating the sensor in an aqueous environmental sample, such as a river, a lake, surface water, or some other body of water. In some examples, prior to incubating the sensor in the aqueous environment, the sensor is incubated in an electrolyte solution for a length of time, as described below with respect to FIG. 5.

As mentioned above, the sensor may be manufactured such that the polymer layer is resistant to collapsing or degrading in an aqueous environment. Accordingly, in some examples the sensor (e.g., the working electrode and/or the polymer layer) is operable to detect a plurality of PFAS after exposing the sensor to an aqueous sample environment. For instance, the polymer layer of the sensor may be stable in the aqueous sample environment for a period of time, such as at least 30 minutes, at least 2 hours, at least 6 hours, or at least 12 hours. In some examples, the affinity sites in the polymer layer do not collapse or change structure after incubation in the aqueous sample environment. In one or more examples, the sensor may be able to detect PFAS after exposing the sensor to an aqueous sample environment for at least 5 minutes, at least 10 minutes, at least 20 minutes, at least 30 minutes, at least 1 hour, at least 2 hours, at least 6 hours, or at least 12 hours.

The sensor may be incubated in the sample of interest for a sufficient time to allow PFAS in the sample of interest to bind to one or more of the plurality of affinity sites. For instance, in some examples the sensor is incubated in the sample of interest for at least 1 minutes, for at least 2 minutes, for at least 5 minutes, for at least 10 minutes, or for at least 20 minutes. In some examples, the sensor may be incubated in the sample of interest for a greater length of time, such as at least 30 minutes, at least 1 hour, at least 2 hours, at least 6 hours, or at least 12 hours.

In one or more examples, the method 300 may comprise a step 304, wherein the PFAS sensor is exposed to a sensing solution different from the sample of interest. Step 304 may be executed by transferring the sensor from an aqueous sample environment containing PFAS to a sensing solution, which may be located in a separate container, such as a vial or a sensing container. The sensing solution may include an electron mediator, such as ferrocenecarboxylic acid (FcCOOH) and a buffer. In one or more examples, the electron mediator is another ferrocene compound or oxygen. In one or more examples, the sensing solution also includes a salt and/or an electrolyte. In one or more examples, the remaining detection steps of method 300 are conducted while the working electrode and/or polymer layer are in contact with (e.g., exposed to or submerged in) the sensing solution.

In one or more examples, method 300 may comprise a step 306, wherein a plurality of PFAS molecules that have bonded to one or more of the plurality of PFAS affinity sites may be detected. In one or more examples, the plurality of PFAS molecules have different molecular structures than a template PFAS used in fabrication of the affinity sites. Step 306 may be executed after an electrode upon which the PFAS affinity sites are disposed is placed in an environment that is potentially contaminated with PFAS (e.g., a river or other surface water). As described at step 304, in one or more examples, step 306 may be executed after exposing the electrode to a sensing solution. The plurality of PFAS molecules may be detected by an electrode (e.g., electrode 204 of system 200 shown in FIG. 2). In one or more examples, detection of the PFAS molecules by the electrode may be based on a change in a charge-transferring reaction occurring between the electrode and the surrounding environment.

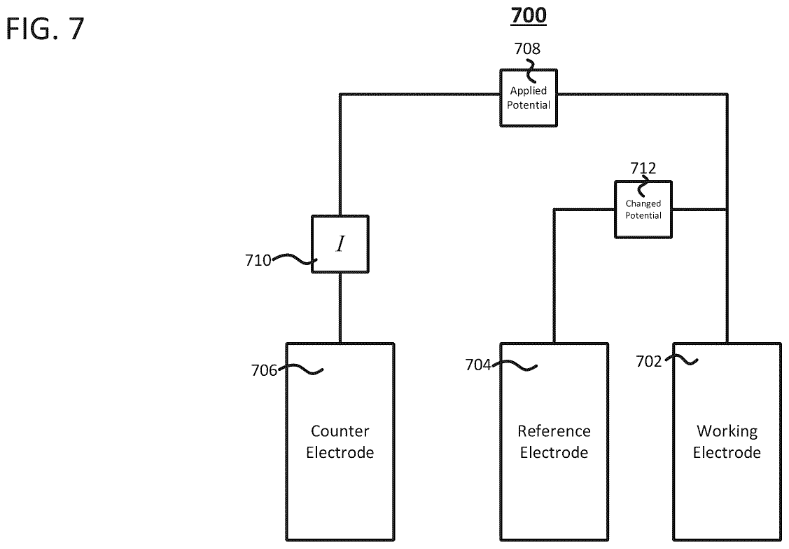

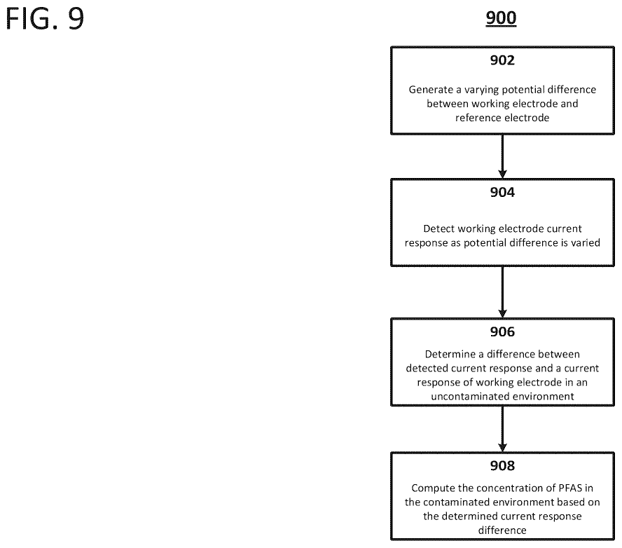

After the plurality of PFAS molecules have been detected in step 306, method 300 may proceed to step 308, wherein a concentration of PFAS in the environment (e.g., in the sample of interest) may be determined. In some examples, determining the concentration of PFAS in the environment is performed by way of one or more of the steps shown in FIG. 9 and described with respect to method 900. For instance, step 308 may be performed using an electrode modified with PFAS affinity sites as a working electrode in a three-electrode system (e.g., as working electrode 702 in three-electrode system 700 shown in FIG. 7).

In one or more examples, the concentration of PFAS may be determined based on the detected plurality of PFAS molecules that are bonded to the one or more PFAS affinity sites. In one or more examples, the determined concentration of PFAS may comprise a total concentration of all PFAS having different molecular structures that can bind to the one or more PFAS affinity sites. However, in other examples the determined concentration of PFAS may comprise a concentration of a particular PFAS species that can being to one or more of the PFAS affinity sites. In one or more examples, a system configured to execute method 300 may be configured to provide users with a list of PFAS that may be contributing to the determined PFAS concentration. In one or more examples, the determined PFAS concentration can be used to develop a calibration curve for quantifying an amount of PFAS in the environment.

In one or more examples, a system configured to execute method 300 may be capable of measuring PFAS concentrations as low as about 1000 parts-per-trillion (ppt), about 500 ppt, about 200 ppt, about 100 ppt, about 50 ppt, about 20 ppt, about 10 ppt, about 9 ppt, about 8 ppt, about 7 ppt, about 6 ppt, about 5 ppt, about 4 ppt, about 3 ppt, about 2 ppt, about 1 ppt, about 0.5 ppt, or about 0.2 ppt. In one or more examples, a system configured to execute method 300 may be capable of measuring PFAS concentrations greater than or equal to about 0.1 ppt, 0.5 ppt, 0.75 ppt, 1 ppt, 2 ppt, 3 ppt, 4 ppt, 5 ppt, 10 ppt, 20 ppt, 50 ppt, 100, pppt, 200 ppt, or 500 ppt. In one or more examples, a system configured to execute method 300 may be capable of measuring PFAS concentrations lower than 1000 ppt, 500 ppt, 200, ppt, 100, ppt, 50 ppt, 20 ppt, 10 ppt, 9 ppt, 8 ppt, 7 ppt, 6 ppt, 5 ppt, 4 ppt, 3 ppt, 2 ppt, or 1 ppt.

### PFAS Affinity Sites

As explained in the preceding section, an electrode may be modified with a polymer layer having affinity sites for detecting a plurality of PFAS molecules. In one or more examples, each affinity site may be created using a template PFAS by polymerizing the polymer layer around the chosen template PFAS (e.g., PFOS). Affinity sites may be configured to have a high selectivity for a molecular structure that appears in the template PFAS. While PFAS that are different from the template PFAS will have different overall molecular structures, one or more PFAS that are different from the template PFAS may contain molecular structures that are identical to or nearly identical to the molecular structure that appears in the template PFAS (i.e., the molecular structure for which the affinity sites have a high affinity). As a result, the one or more PFAS that are different from the template PFAS but contain the identical (or nearly identical) molecular structure for which the affinity sites select may also be able to bind to the affinity site. In some examples, he affinity sites may be able to bind to greater than two PFAS that are different from the template PFAS, or a greater number (e.g., three or more, five or more, 10 or more, or 20 or more) of PFAS different from the template PFAS. In some examples, the affinity sites are able to bing to an entire class of PFAS, the class optionally encompassing the template PFAS. A single modified electrode may therefore be capable of measuring concentrations of multiple different PFAS at the same time.

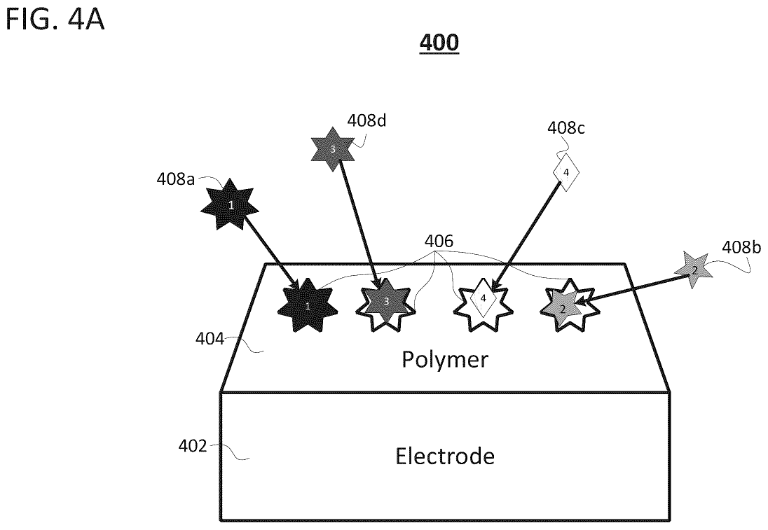

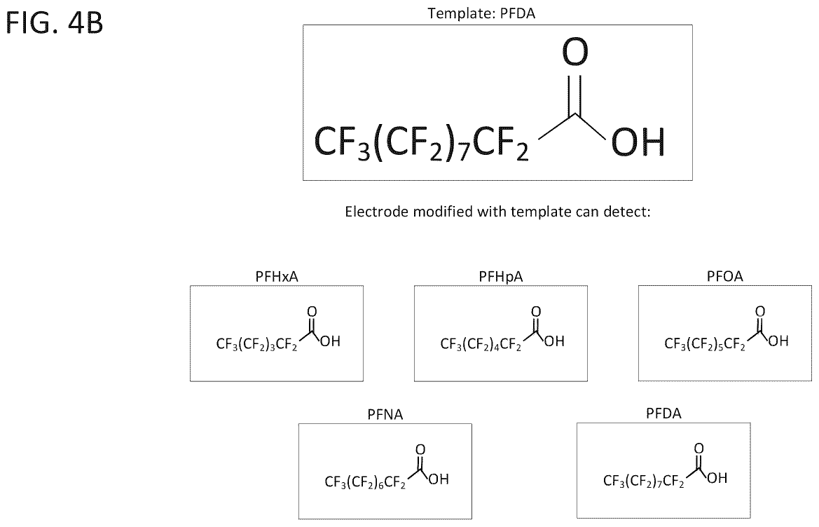

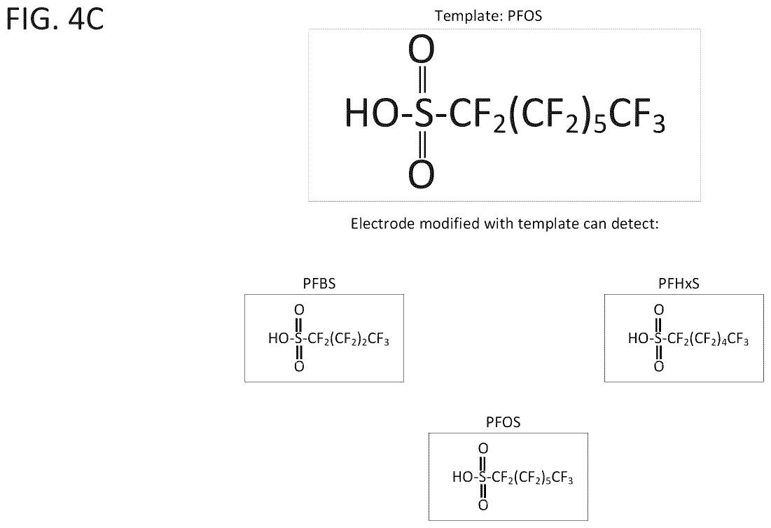

FIG. 4A illustrates exemplary binding behavior of PFAS affinity sites, according to one or more examples of the present disclosure. Specifically, FIG. 4A illustrates a modified electrode system 400 comprising an electrode 402 and a polymer 404 containing a plurality of PFAS affinity sites 406. In one or more examples, modified electrode system 400 may be a component of a system for quantifying amounts of PFAS (e.g., system 200 shown in FIG. 2). In one or more examples, electrode 402 may include one or more features of electrode 204 of system 200 shown in FIG. 2. In one or more examples, polymer 404 may include one or more features of polymer 206 of system 200 shown in FIG. 2. In one or more examples, PFAS affinity sites 406 may include one or more features of affinity sites 208 of system 200 shown in FIG. 2.

In one or more examples, PFAS affinity sites 406 may be cavities in polymer 404 that have a high affinity for a molecular structure found in a template PFAS. As shown, PFAS affinity sites 406 are configured to have a high affinity for a molecular structure found in template PFAS 408a. In one or more examples, the molecular structure for which PFAS affinity sites 406 have a high affinity may be chosen based on the prevalence of a particular PFAS in an environment of interest and/or based on a degree of scrutiny of molecules of similar structures.

As a result of this high affinity for the molecular structure found in template PFAS 408a, PFAS affinity sites 406 may be configured to selectively form bonds with molecules that either have said molecular structure or have different molecular structures that are similar to said molecular structure. Therefore, in one or more examples, PFAS affinity sites 406—all of which have a high affinity for the molecular structure found in template PFAS 408a—may also bind to PFAS such as PFAS 408b, PFAS 408c, and/or PFAS 408d. PFAS 408b-408d may contain different molecular structures that are at least 10%, at least 20%, at least 30%, at least 40%, at least 50%, at least 60%, at least 70%, at least 80%, or at least 90% identical to the molecular structure contained in template PFAS 408a. In one or more examples, a concentration of PFAS in an environment that is measured using a system comprising modified electrode system 400 may be a total concentration that includes the concentrations of PFAS 408a, PFAS 408b, PFAS 408c, and PFAS 408d.

In one or more examples, PFAS affinity sites 406 may target functional groups of different chain lengths. For instance, in one or more examples, template PFAS 408a may be the PFOS molecule (see FIG. 1A), but PFAS affinity sites 406 may be capable of binding to sulfonic acid PFAS of 8, 7, 6, 5, 4, 3, or 2 carbon chains.

In one or more examples, the specificity of the PFAS affinity sites 406 is controlled at least partially based on the solution used to extract the template PFAS from the polymer 404. For instance, an extraction solution including acetone and an acid may result in PFAS affinity sites having higher specificity, such that the PFAS affinity sites bind to only PFAS identical to or similar to the molecular structure found in the template PFAS. In other examples, an extraction solution including acetone and water may result in PFAS affinity sites having lower specificity, such that they bind to a larger subset of PFAS having different molecular structures than the template PFAS.

FIG. 4B illustrates an exemplary template PFAS and exemplary PFAS that an electrode modified with the template PFAS may be capable of detecting. Specifically, FIG. 4B shows molecules that may be detected by an electrode that has been modified to detect perfluorocarboxylic acids (PFCA). Various PFCA compounds differ by total number of difluoromethylene groups (CF₂) present between the trifluromethyl group (CF₃) and the difluoromethylene bonded to the carboxylic acid group (C(═O)OH). As shown, a working electrode configured to detect a plurality of perfluorocarboxylic acids may be modified using perfluorodecanoic acid (PFDA), which contains seven difluoromethylene groups between the trifluromethyl group and the difluoromethylene bonded to the carboxylic acid group. A working electrode that has been modified using PFDA as a template molecule may be capable of detecting, along with PFDA, perfluorononanoic acid (PFNA), perfluorooctanoic acid (PFOA), perfluoroheptanoic acid (PFHpA), and perfluorohexanoic acid (PFHxA), each of which have molecular structures that are identical to PFDA except with fewer difluoromethylene groups between the trifluromethyl group and the difluoromethylene group to the carboxylic acid group.

FIG. 4C illustrates an exemplary template PFAS and exemplary PFAS that an electrode modified with the template PFAS may be capable of detecting. Specifically, FIG. 4C shows molecules that may be detected by an electrode that has been modified to detect perfluorosulfonic acids (PFSA). Various PFSA compounds differ by total number of difluoromethylene groups (CF₂) present between the difluoromethylene bonded to sulfonic acid group (S(═O)₂(OH)) and the trifluoromethyl group (CF₃). As shown, a working electrode configured to detect a plurality of perfluorosulfonic acids may be modified using perfluorooctanesulfonic acid (PFOS), which contains five difluoromethylene groups between the difluoromethylene bonded to sulfonic acid group (S(═O)₂(OH)) and the trifluoromethyl group (CF₃). A working electrode that has been modified using PFOS as a template molecule may be capable of detecting, along with PFOS, perfluorohexanesulfonic acid (PFHxS) and perfluorobutanesulfonic acid (PFBS), each of have molecular structures that are identical to PFOS except with fewer difluoromethylene groups between the difluoromethylene bonded to sulfonic acid group (S(═O)₂(OH)) and the trifluoromethyl group (CF₃).

### Methods of Manufacturing a PFAS Sensor

As explained in the preceding section, a PFAS sensor can be formed by modifying an electrode with a polymer layer having a plurality of affinity sites for detecting PFAS molecules. Aspects of the present disclosure also relate to improved methods of manufacturing PFAS sensors. PFAS sensors formed by conventional methods may be inoperable in aqueous environments, and particularly aqueous environments containing low levels of PFAS, due to the collapsing of the polymer layer when no PFAS are bound to the affinity sites. The methods of manufacture described herein may advantageously result in more resilient PFAS sensors capable of detecting PFAS after exposure to aqueous environments, even after long periods of incubations in environments containing low levels of PFAS. Further, the manufacturing steps described herein may give users more precise control over the binding characteristics of the PFAS sensor, e.g., in order to create PFAS sensors having affinity sites for specific or non-specific detection of various PFAS.

FIG. 5 illustrates an exemplary method for manufacturing a PFAS sensor by modifying an electrode with PFAS affinity sites according to one or more examples of the present disclosure. Specifically, FIG. 5 shows a method 500 for modifying a working electrode with PFAS affinity sites using molecular imprinting techniques. In one or more examples, method 500 may be used to create a modified electrode system such as modified electrode system 400 shown in FIG. 4A.

In one or more examples, method 500 may comprise a step 502, wherein an electrode may be prepared for modification. Preparing an electrode for modification may comprise polishing the electrode with alcohol (e.g., ethanol) and/or silica, rinsing the electrode with water (e.g., nanopure water) one or more times, and/or subjecting the electrode to ultrasonic vibrations. In one or more examples, the electrode is polished using a polishing pad (e.g., with grain alumina of 0.05 μm) to dislodge and abrade any unwanted products on the electrode. In one or more examples, the electrode is sonicated for about 5 minutes, for about 4 minutes, for about 3 minutes, for about 2 minutes, for about 1 minute, for less than 1 minute, or for greater than 5 minutes. In one or more examples, step 502 may be performed if an electrode that has previously been modified needs to be re-modified, e.g., in order to dislodge any previous MIPs formed on the electrode.

After the electrode is prepared for modification in step 502, method 500 may proceed to step 504, wherein the un-modified electrode may be characterized. In one or more examples, characterizing the un-modified electrode may provide information related to the active portions of the electrode's surface area. In one or more examples, characterizing the un-modified electrode may comprise measuring behaviors of the electrode in a controlled environment. In one or more examples, characterizing the un-modified electrode may comprise placing the electrode in an acid solution (e.g., a sulfuric acid solution or a hydrochloric acid solution, such as a solution of 0.1 M H₂SO₄) and then measuring a current response of the electrode as a signal with a varying potential is applied to the electrode. In one or more examples, characterizing the un-modified electrode may comprise placing the electrode in a faradic solution and then measuring a current response of the electrode as a signal with a varying potential is applied to the electrode. In one or more examples, characterizing the un-modified electrode may comprise placing the electrode in a solution comprising a fast, one-electron mediator (e.g., a solution of ferrocene methanol or a solution of ferrocene carboxylic acid) and then measuring a current response of the electrode as a signal with a varying potential is applied to the electrode. In one or more examples, the electrode may be characterized after a certain number of uses of the electrode to detect PFAS, such as after every use, after every two uses, after every three uses, after every five uses, or after every ten uses. In one or more examples, the electrode may be repeatedly acid washed and characterized until reduction peaks overlap. In one or more examples, the electrode may be repeatedly acid washed and characterized until a shift has occurred in the gold oxide peak of the electrode is observed and/or until an area under the gold oxide peak is greater than a predetermined value, such as about 30 pf. In one or more examples, after characterizing the electrode in step 504, the electrode is rinsed with water (e.g., nanopure water), optionally until the water runoff is greater than a certain pH (e.g., greater than 5.0, greater than 5.5, greater than 6, or greater than 6.5). In one or more examples, after the un-modified electrode is characterized in step 504, method 500 may proceed to step 506, wherein a polymer layer is formed on the working electrode. In some examples, the polymer layer may be formed on a surface of the electrode by polymerizing a plurality of monomers to form the polymer layer around a plurality of template PFAS molecules. The plurality of monomers may be caused to self-assemble around the template PFAS molecules due to interactions between the template PFAS and the monomers. In one or more examples, the self-assembled monomers may be polymerized to form a molecular imprinted polymer (MIP). This may trap the template PFAS within the polymer layer on the surface of the electrode.

In one or more examples, forming the polymer layer on the working electrode comprises contacting the working electrode with a solution comprising a plurality of monomers and a plurality of template PFAS. For example, the solution may be poured over the working electrode such that a surface of the working electrode is coated or submerged in the solution. In one or more examples, the solution is an acetone solution (i.e., includes acetone). In some examples, the solution is a methanol solution (i.e., includes methanol). In some examples, the monomers are ortho-phenyldiamine (o-PD) monomers. However, additional or alternative monomers may also be included in the solution and polymerized to form the polymer layer.

In one or more examples, the template PFAS is a single PFAS, such as any of the PFAS mentioned above. However, in other examples the template PFAS includes two or more PFAS, e.g., in order to create a polymer layer having affinity sites formed from various template PFAS for less selective binding and more robust detection of multiple PFAS. In some examples, the solution is sonicated to mix the template PFAS and the monomers in the solution prior to initiating polymerization.

In one or more examples, the solution includes an acid, such as hydrochloric acid (HCl). The presence of acid in the solution during self-assembly of the polymer layer may cause increased polymer chain stiffness, cross-linking, covalent bonding, and/or non-covalent interactions between the plurality of monomers, resulting in a more mechanically robust polymer layer that is less likely to collapse or degrade when exposed to aqueous environments. In a particular example, polymerization in an acidic solution causes an increased number of ring structures, increased pi-pi stacking, and/or increased semicrystalline physical crosslinking in the polymer layer compared with polymerization in more basic solutions, resulting in a more mechanically robust polymer layer. In one or more examples, the acid may be added to the solution following the addition of the plurality of monomers and the plurality of template PFAS. Optionally, an amount of water is added to the solution prior to the addition of the acid. In some examples, the solution, including the acid, has a pH of about 2.2. In one or more examples, the pH of the solution may be between 1 and 3, between 1.5 and 2.5, between 2 and 3, between 2 and 2.5, or between 2 and 2.2. In one or more examples, the solution may have a pH that is less than 3, less than 2.5, or less than 2.

In one or more examples, forming the polymer layer includes polymerizing the monomers to form the polymer layer and trap the template PFAS in the polymer layer. In some examples, the polymer layer is formed by electropolymerizing the monomers. For example, the monomers may be electropolymerized by applying one or more voltage cycles to the monomers in the solution, such as 10 mV/s for about 15 cycles. However, higher or lower voltages may be applied (e.g., 5 mV/s to 10 mV/s, 10 mV/s to 15 mV/s, 15 mV/s to 20 mV/s, or greater than 20 mV/s) for greater or fewer cycles (e.g., 5 to 10 cycles, 10 to 15 cycles, 15 to 20 cycles, or greater than 20 cycles) to electropolymerize the monomers and form the polymer layer.

In one or more examples, after forming the polymer layer in step 506, the polymer layer is characterized in a ferrocene solution, such as a ferrocene methanol solution or a ferrocene carboxylic acid solution. In one or more examples, after forming and/or characterizing the polymer layer, the working electrode is rinsed with water (e.g., nanopure water), optionally until the water runoff is greater than a certain pH (e.g., greater than 5.0, greater than 5.5, greater than 6, or greater than 6.5). In one or more examples, after forming the polymer later, the reference electrode and/or counter electrode are rinsed with an acetone solution.

In one or more examples, after the electrode is modified with the polymer layer in step 506, method 500 may proceed to step 508, wherein the PFAS template molecules may be extracted from the polymer layer to create a plurality of affinity sites in the polymer layer. In one or more examples, after the PFAS template molecules have been removed, the polymer layer may comprise a plurality of cavities having complementary structures to a molecular structure contained in the PFAS template. The plurality of cavities may form the PFAS affinity sites.

In one or more examples, extracting the plurality of template PFAS comprises washing the polymer layer with a solution or incubating the polymer layer in a solution. In one or more examples, the template PFAS can be extracted with a solution including acetone and/or water (e.g., a mixture of water and 0-100% acetone), such as a 1:1 acetone:water solution. Extraction of the PFAS templates with an acetone solution may result in the creation of non-specific affinity sites in the polymer layer that are configured to bind to multiple PFAS (e.g., one or more PFAS having molecular structures that are different from the molecular structure of the template PFAS). In one or more examples, the template PFAS can be extracted with a solution including acetone and/or an acid (e.g., a mixture of an acid and 0-100% acetone), such as a 1:1: acetone:acid solution. In one or more examples, the acid is hydrochloric acid and/or sulfuric acid, and optionally the acid diluted with water. Extraction of the PFAS templates with an acidic acetone solution may result in the creation of specific affinity sites in the polymer layer that are configured to bind to a single PFAS or a limited number of PFAS (e.g., one or more PFAS having molecular structures that are the same or similar as a molecular structure of PFAS). Accordingly, the composition of the extraction solution may impact the binding specificity of the affinity sites, allowing a user to selectively create a PFAS sensor that is specific to (i.e., configured to bind to and then detect) only a particular desired subset of PFAS. In one or more other examples, the template PFAS can be extracted with a solution including methanol and/or water (e.g., a mixture of water and 0-100% methanol), such as a 1:1 methanol:water solution. In additional or alternative examples, the template PFAS can be extracted with a solution including a mixture of the three (e.g., a mixture including water, methanol and/or acetone in any desired ratio).

In one or more examples, extracting the template PFAS includes washing and/or incubating the polymer layer for at least 5 minutes, at least 10 minutes, at least 20 minutes, or at least 30 minutes. In one or more examples, extraction of the polymer layer is conducted without stirring the solution. In one or more examples, after extracting the template PFAS at step 508, the polymer layer may be characterized. In some examples, the polymer layer is characterized in a ferrocene solution, such as a ferrocene methanol solution or a ferrocene carboxylic acid solution.

In one or more examples, after the PFAS template molecules have been extracted from the polymer layer step 508, method 500 may proceed to step 510, wherein the working electrode and the polymer layer are incubated in a solution, such as an electrolyte solution. Incubating the working electrode and the polymer layer in the solution may cause the solution to saturate and condition the polymer layer. In some examples, the working electrode and polymer layer are incubated overnight, such as incubated for at least 12 hours. However, in other examples, the working electrode and polymer layer may be incubated in the electrolyte solution for greater or lesser amounts of time, such as at least 2 hours, between 2 hours and 4 hours, between 4 hours and 6 hours, between 6 hours and 8 hours, between 8 hours and 10 hours, between 10 hours and 12 hours, between 12 and 24 hours, or greater than 24 hours. In one or more examples, the PFAS sensor may be incubated in the electrolyte solution immediately prior to its use for detecting an amount of PFAS.

In one or more examples, the electrolyte solution is an ammonia solution (i.e., a solution including ammonia). However, other electrolyte solutions may also be used, such as solutions including phosphate and/or potassium. The pH of the solution may be relatively high, such as about 8.4 pH. In one or more examples, the pH of the electrolyte solution may have a pH of at least 6, a pH of at least 7, a pH of at least 8, or a pH of at least 8.5. In one or more examples, the pH of the electrolyte solution is between 7 and 8, between 8 and 9, between 7 and 7.5, between 7.5 and 8, between 8 and 8.5, between 8.5 and 9, or greater than 9.

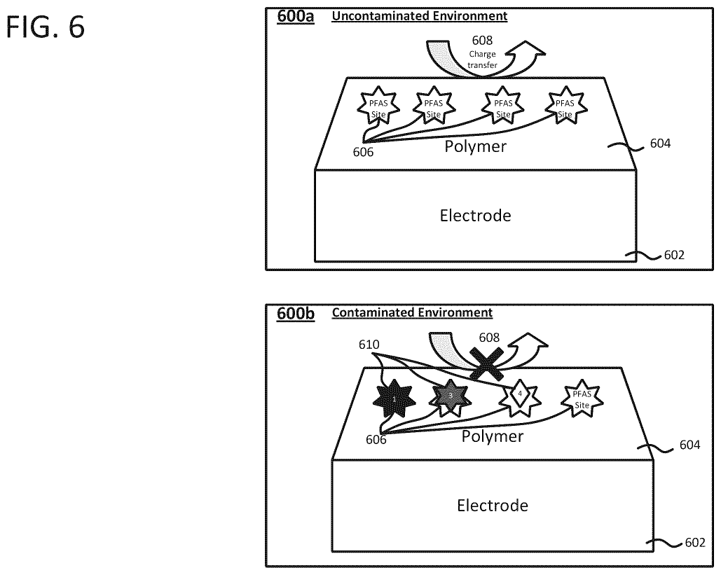

FIG. 6 illustrates exemplary responses in an uncontaminated and in a contaminated environment of a PFAS sensor including an electrode that is modified with PFAS affinity sites, according to one or more examples of the present disclosure. Specifically, FIG. 6 shows responses of an electrode 602 that is modified with a polymer 604 comprising a plurality of PFAS affinity sites 606 in an uncontaminated environment 600a and in a contaminated environment 600b. Electrode 602 may be a component of a system for quantifying amounts of PFAS (e.g., system 200 shown in FIG. 2).

Uncontaminated environment 600a may contain PFAS at concentrations below the sensitivity of electrode 602—i.e., may contain PFAS in such low concentrations that no PFAS molecules bind to PFAS affinity sites 606. As such, PFAS affinity sites 606 may be unblocked cavities that leave a certain portion of the surface area of electrode 602 exposed to uncontaminated environment 600a. In one or more examples, when a voltage is applied to electrode 602, a charge-transferring reaction 608 may be induced between the exposed portions of electrode 602 and one or more substances in uncontaminated environment. Charge-transferring reaction 608 may induce a measurable response in electrode 602 that depends on the applied voltage.

Contaminated environment 600b may contain a concentration of PFAS that is high enough to be detected by electrode 602. Thus, when electrode 602 is placed in contaminated environment 600b, a plurality of PFAS molecules may bind to one or more PFAS affinity sites 606, thereby blocking previously exposed portions of the surface area of electrode 602. When a voltage is applied to electrode 602, charge-transferring reaction 608 may be reduced or entirely prevented, thereby altering the measurable, voltage dependent response of electrode 602. In one or more examples, the response of electrode 602 in contaminated environment 600b may be compared to the response of electrode 602 in uncontaminated environment 600a in order to determine a total concentration of PFAS in contaminated environment 600b.

### Detection of PFAS and Determination of Concentrations

When a voltage is applied to an electrode, a charge-transferring reaction may be induced between the electrode and the surrounding environment. If this electrode is coupled to a larger circuit, the charge-transferring reaction may generate a current in that circuit. The current that is generated may depend on the voltage that is applied to the electrode. However, when PFAS molecules bind to affinity sites on a modified electrode (e.g., as shown in FIG. 6), they may reduce the total electrode surface area that is exposed to the surrounding environment. Thus, if a voltage is applied to an electrode modified with affinity sites that have bonded to PFAS molecules, the charge-transferring reaction may occur to a lesser degree, or, in some cases, may not occur at all. The current response of the modified electrode at a given voltage may decrease as a result. In one or more examples, changes in electrode behavior (e.g., changes in current response at a given voltage or over a given voltage range) between uncontaminated environments and contaminated (i.e., PFAS-containing) environments may be used to determine concentrations of PFAS in the contaminated environment. Measuring the change in electrode behavior in one or more PFAS-containing environments (e.g., a plurality of environments containing varying amounts of PFAS) may enable the creation of calibration curves for the estimation and quantification of the amount of PFAS in environments containing an unknown amount of PFAS. For instance, determining the typical electrode behavior, such as a typical current decay pattern, at a particular PFAS concentrations may allow a user of the sensor to correlate that particular electrode behavior with a particular concentration of one or more PFAS in an environment.

FIG. 7 illustrates an exemplary three-electrode system for measuring a current response of a modified electrode, according to one or more examples of the present disclosure. Specifically, FIG. 7 shows a three-electrode system 700 comprising a working electrode 702, a reference electrode 704, and a counter electrode 706. In one or more examples, working electrode 702 may be a component (e.g., electrode 202) of a PFAS detection system (e.g., system 200). Working electrode 702 may be coupled to reference electrode 704 and counter electrode 706 so that one or more behaviors of working electrode 702 may be accurately quantified and/or measured. Said quantified behaviors may then be used to determine a concentration of PFAS in an environment.

Working electrode 702 may be configured to respond to a concentration of a class of PFAS molecules in an environment (e.g., in a body of water). In one or more examples, working electrode 702 may be modified with a molecularly imprinted polymer (MIP) layer comprising a plurality of PFAS affinity sites.

Reference electrode 704 may be configured to have a stable and well-known electrode potential. In one or more examples, reference electrode 704 may be configured to pass no current. In one or more examples, reference electrode 704 may comprise silver or silver chloride. In one or more examples, reference electrode 704 may be a pseudo-reference electrode such as a bare silver wire.

In one or more examples, physical properties such as electrode potential of reference electrode 704 may not be well-defined. In one or more examples, PFAS in an environment may be detected by measuring a magnitude or a change in magnitude of a current response at working electrode 702.

Counter electrode 706 may be configured to balance reactions that occur at working electrode 702 and complete the three-electrode circuit. For example, if working electrode 702 is an anode, counter electrode 706 may be a cathode (or vice versa). In one or more examples, counter electrode 706 may comprise a conductive surface. The conductive surface may comprise silver, platinum, or graphite. In one or more examples, the surface area of counter electrode 706 may be larger than the surface area of working electrode 702.

In one or more examples, behaviors of working electrode 702 in an environment that is contaminated with PFAS may be determined by exposing three-electrode system 700 to the environment and subsequently applying a known potential 708 between working electrode 702 and counter electrode 706. Potential 708 may induce charge-transferring reactions at working electrode 702. The charge-transferring reactions may cause a change 712 in potential between working electrode 702 and reference electrode 704. Since reference electrode 704 may be configured to have a stable electrode potential and not to pass current, the changed potential 712 must necessarily be caused by a change in a current response of working electrode 702. This current response 710 may be measured and used to compute a PFAS concentration. In one or more examples, electrical signals between working electrode 702, reference electrode 704, and/or counter electrode 706 may be controlled and/or measured using a potentiostat.

In one or more examples, a current response of a modified electrode may be measured using a two-electrode system, rather than a three-electrode system. A two-electrode system may comprise a modified electrode such as working electrode 702 and a second electrode that functions as both a reference electrode and a counter electrode (e.g., a second electrode that performs the functions of both reference electrode 704 and counter electrode 706). In one or more examples, the potential difference may be varied between the modified (working) electrode and the second electrode. The second electrode may have a known electrode potential and may be configured to balance the reaction that occurs at the modified (working) electrode.

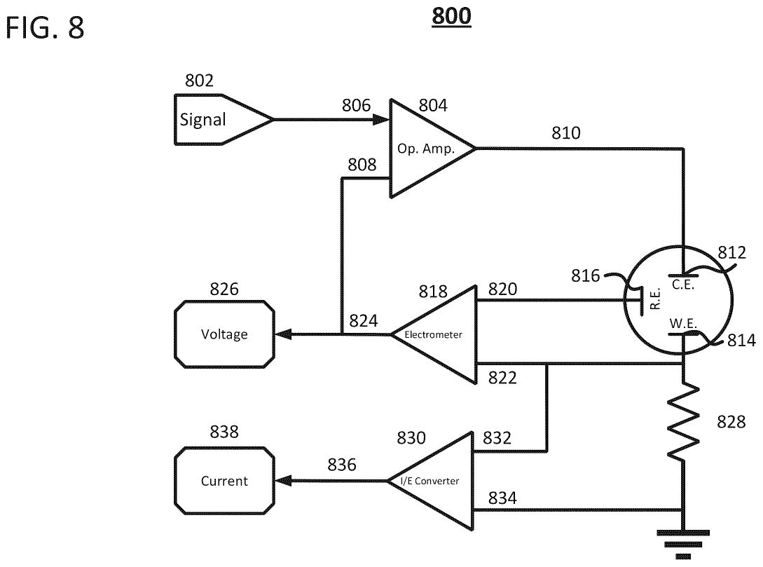

FIG. 8 illustrates an exemplary potentiostat for controlling a potential difference between a reference electrode and a working electrode, according to one or more examples of the present disclosure. Specifically, FIG. 8 illustrates a potentiostat 800 configured to control a potential difference between a reference electrode and a working electrode. Potentiostat 800 may be a component of a control system of a PFAS quantification system (e.g., control system 202 of system 200 shown in FIG. 2). In one or more examples, the working electrode that is coupled to potentiostat 800 may be a component of a PFAS quantification system (e.g., electrode 204 of system 200 shown in FIG. 2).

In one or more examples, an input signal 802 may be received. Input signal 802 may be generated by a user using a signal generator. In one or more examples, input signal 802 may be transmitted to a first input 806 of an operational amplifier 804. A second operational amplifier input 808 may be a signal with a voltage equal to the potential difference between the working electrode and the reference electrode.

In one or more examples, operational amplifier 804 may output a signal 810 having a voltage equal to the potential difference between the signal received at first operational amplifier input 806 and the signal received at second operational amplifier input 808. Signal 810 may be applied to a counter electrode lead 812 coupled to a counter electrode and may induce charge-transferring reactions at the working electrode.

In one or more examples, a reference electrode lead 816 coupled to the reference electrode and a working electrode lead 814 coupled to the working electrode may be configured to transmit signals to a first input 820 and a second input 822, respectively, of an electrometer 818. Electrometer 818 may output a signal 824 having a voltage equal to the potential difference between the working electrode and the reference electrode. Signal 824 may be transmitted to second operational amplifier input 808. In one or more examples, signal 824 may be transmitted to a voltmeter 826 so that the potential difference induced between the working electrode and the reference electrode by input signal 802 may be measured.

In one or more examples, working electrode lead 814 may be coupled to a resistor 828. Resistor 828 may have a known resistance. Working electrode lead 814 may transmit a signal 822 to a first input 832 of an TIE converter 830. A second input 834 of UE converter 830 may be coupled to an end of resistor 828 that is not directly connected to working electrode lead 814. I/E converter 830 may output a signal 836 having a voltage equal to the voltage drop across resistor 828 as a result of the current response of the working electrode. Signal 830 may be transmitted to a processor configured to compute the current based on the resistance of resistor 828 and the voltage of signal 830. This current may be used to determine a concentration of PFAS in an environment.

FIG. 9 illustrates an exemplary method for determining a concentration of PFAS using an electrode modified with PFAS affinity sites (i.e., the “PFAS sensor”), according to one or more examples of the present disclosure. Specifically, FIG. 9 shows a method 900 for determining a concentration of PFAS by using a modified electrode as a working electrode in a three-electrode system (e.g., as working electrode 702 in three-electrode system 700 shown in FIG. 7).

In one or more examples, and as explained with respect to step 304 of method 300 above, the determination of a concentration of PFAS using the PFAS sensor may be conducted in a sensing solution, which may include an amount of PFAS. In one or more examples, the sensing solution contains an electron mediator such as ferrocene carboxylic acid (i.e., FcCOOH), oxygen, or ferrocene methanol (e.g., C₁₁H₁₂FeO). In one or more examples, the sensing solution includes an amount of salt. In one or more examples, the method 900 is conducted immediately after the PFAS sensor is incubated in an electrolyte solution as described in step 510 of the method 500 shown in FIG. 5. For instance, method 900 may include, prior to proceeding to step 902, removing the PFAS sensor from an electrolyte solution and exposing the sensor to a sensing solution.

In one or more examples, method 900 may comprise a step 902, wherein, step 902 includes generating a varying potential difference between the working electrode (i.e., the electrode that is modified with a plurality of PFAS affinity sites) and a reference electrode. In some examples, step 902 may be conducted directly in the contaminated sample environment. Optionally, oxygen or some other electron mediator may be added to the sample environment before generating the potential difference between the electrodes. In one or more other examples, step 902 may be conducted after placing the sensor in a sensing solution (e.g., the sensing solution described above and with respect to step 304 of method 300) following incubation of the sensor in the sample environment. In one or more examples, the varying potential difference may be generated using a signal generator and/or a potentiostat (e.g., potentiostat 800 shown in FIG. 8). The range over which the potential difference between the working electrode and the reference electrode is varied may depend on one or more environmental conditions, such as the environmental pH. In one or more examples, the potential difference between the working electrode and the reference electrode may be varied between about −1-0V, about −1-0.2 V, about −1-0.4 V, about −1-0.6 V, about −1-0.8 V, about −1-1.0 V, about −1-2.0 V, about −1-5.0V, about 0-0.2 V, about 0-0.4 V, about 0-0.6 V, about 0-0.8 V, about 0-1.0 V, about 0-2.0 V, or about 0-5.0 V.

While a varying potential difference between the working electrode and the reference electrode is being generated at step 902, method 900 may proceed to a step 904, wherein a current response of the working electrode may be detected. In one or more examples, the current response of the working electrode may be detected by measuring a voltage drop across a known resistor that is connected to the working electrode. In one or more examples, the current response may be measured using a potentiostat (e.g., potentiostat 800 shown in FIG. 8).

In one or more examples, after the current response of the working electrode is measured as the potential difference is varied at step 904, method 900 may proceed to a step 906, wherein the detected current response may be compared to a current response of the working electrode in an uncontaminated environment and/or to a pre-programmed calibration curve. Specifically, differences between the detected current response in the contaminated environment and the uncontaminated current response may be determined and quantified.

After the differences between the contaminated current response and the uncontaminated current response have been determined at step 906, method 900 may proceed to step 908, wherein a concentration of PFAS in the contaminated environment may be determined based on the determined differences in current responses.

### Detecting Multiple Classes of PFAS

As described in the preceding sections, an electrode modified with PFAS affinity sites that were created using a template PFAS may be used to determine the concentration of a class of PFAS having overlapping or similar molecular structures to a molecular structure found in the template PFAS. In other words, the electrode may be associated with the class of PFAS. In one or more examples, a system for quantifying amounts of PFAS in an environment (e.g., system 200 shown in FIG. 2) may comprise a second electrode that is modified with a second polymer layer having a second plurality of PFAS affinity sites for detecting a second plurality of PFAS molecules. The second plurality of PFAS affinity sites may be generated using a second template PFAS, which may be different from a first template PFAS of a first electrode. Thus, the second electrode may be associated with a second class of PFAS, which may be different from a first class of PFAS. Each PFAS in the second class of PFAS may have molecular structures that overlap or are similar to a molecular structure found in the second template PFAS. The system may similarly comprise a third modified electrode associated with a third class of PFAS, a fourth modified electrode associated with a fourth class of PFAS, and so on. In one or more examples, a system comprising multiple modified electrodes may be capable of measuring concentrations of large numbers of PFAS in a single measurement session.

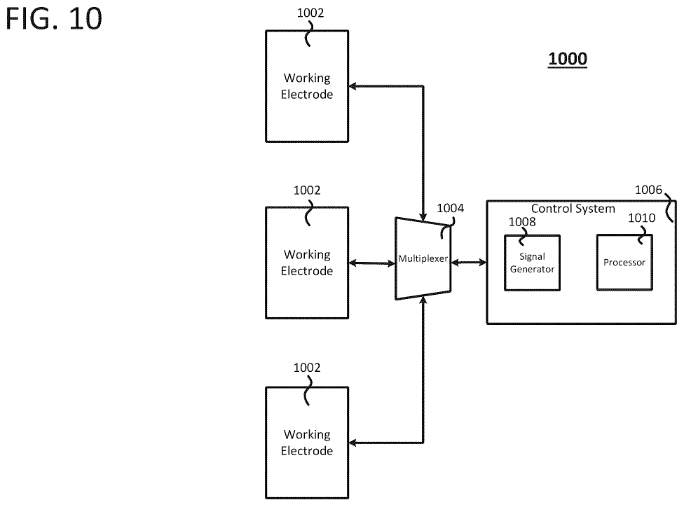

FIG. 10 illustrates an exemplary system for quantifying amounts of multiple classes of PFAS associated with multiple modified electrodes, according to one or more examples of the present disclosure. Specifically, FIG. 10 shows a system 1000 comprising a plurality of modified working electrodes 1002, a multiplexer 1004 coupled to each working electrode 1002, and a control system 1006 comprising a signal generator 1008 and a processor 1010. Each working electrode 1002 may include one or more features of electrode 204 shown in FIG. 2, electrode 402 shown in 4, or electrode 602 shown in FIG. 6. In one or more examples, each working electrode 1002 may comprise a plurality of PFAS affinity sites that have an affinity for a molecular structure of a PFAS template. In one or more examples, the PFAS affinity sites of each working electrode 1002 may be capable of binding to multiple PFAS molecules that are different from, but contain similar molecular structures to, the PFAS template used to form said affinity sites. As such, each working electrode 1002 may be configured to respond to the presence of a plurality of different PFAS in a given environment.

In one or more examples, signal generator 1008 may be configured to control voltages that are applied to the plurality of working electrodes 1002. Signal generator 1008 may comprise one or more potentiostats. Processor 1010 may be configured to control signal generator 1008 and/or to analyze signals received from working electrodes 1002 in order to determine concentrations of PFAS measured by each electrode 1002. In one or more examples, multiplexer 1004 may be configured to route signals between the plurality working electrodes 1002 and the control system 1006.

In one or more examples, system 1000 may comprise at least 2, at least 3, at least 4, at least 5, at least 10, at least 15, at least 25, at least 50, or at least 75 working electrodes 1002. In one or more examples, system 1000 may have between 50-55, 55-60, 60-65, 65-70, 70-75, or 75-80 working electrodes 1002. In one or more examples, each working electrode 1002 may be capable of detecting at least 2, at least 10, at least 20, at least 30, at least 40, at least 50, at least 60, at least 70, at least 80, at least 90, at least 100, or at least 1000 different PFAS. Thus, in one or more examples, system 1000 may be capable of determining concentrations of over 1000 different PFAS.

### Example: MIP Fabrication on a Macro Electrode

In one example of the present disclosure, a MIP was fabricated on a macro-electrode, for instance, in order to manufacture a PFAS sensor or sensing system as described above.

### Electrode Preparation

The working electrode was prepared via standard electrochemical practice including polishing and cycling in dilute sulfuric acid.

### FcCOOH Characterization

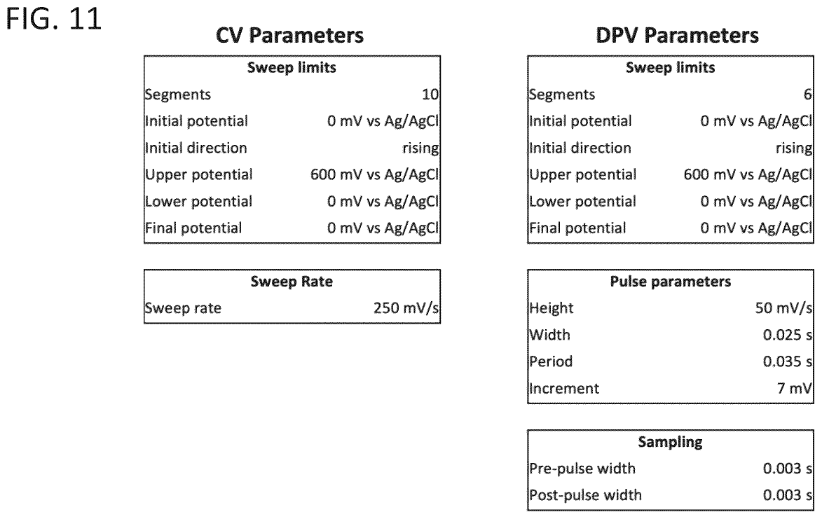

The bare WE was characterized via cyclic volatammetry (CV) and differential pulse voltammetry (DPV) using a solution of 1 mM FcCOOH+0.1 M ammonia at pH 8.4 (sensing solution). The data can be used to identify issues with the electrode or electrode surface prior to MIP preparation. In CV, the voltage is swept from low to high and back again while measuring the current response of the electrode. In DPV, a background CV is taken before the sample CV to provide background substraction and amplify the signal. This is accomplished by applying a linear ramp potential. The CV and DPV measurements were taken using the exemplary scan parameters shown in FIG. 11.

### MIP Fabrication

1 mL of 150 mM ortho-phenylenediamine (o-PD) (81 mg in 5 mL acetone) and 1.5 mL of 10 mM PFOS (538 mg in 100 mL acetone) were mixed and sonicated for 5 minutes. Following sonication, 12 mL of nanopure water and 200 μL of 1M HCl was added to the o-PD/PFOS solution. The resulting solution was gently agitated for up to 5 minutes, allowing for any bubbles to dissipate.

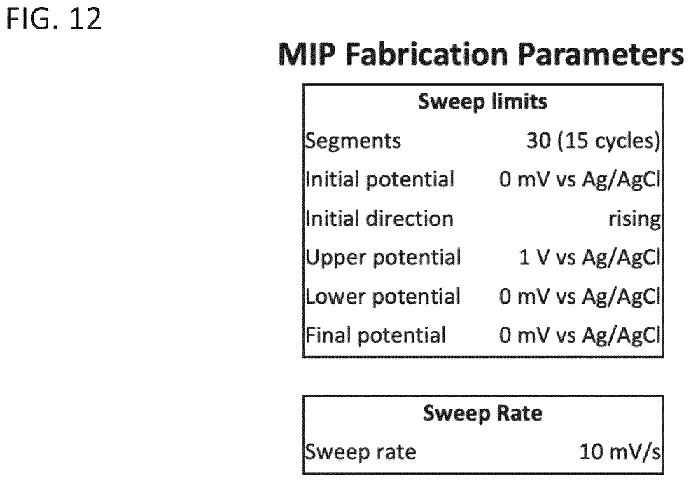

The electrodes were then placed into the solution prepared above (MIP fabrication solution). A MIP fabrication CV was performed. In the scans, current should decay very quickly after the first few scans. Potential was applied for approximately one hour. The WE was then rinsed with nanopure water and placed into a fresh solution of nanopure water for overnight storage. The WE had an orange sheen to it, visually indicating a successful MIP fabrication. Exemplary scan parameters for MIP fabrication are shown in FIG. 12.

### MIP Fc Characterization

Following fabrication of the MIP as described above (“MIP Fabrication”), the electrodes were characterized utilizing ferrocene carboxylic acid or another appropriate electron mediator utilizing CV and DPV as described above (“FcCOOH Characterization”).

### Template Extraction

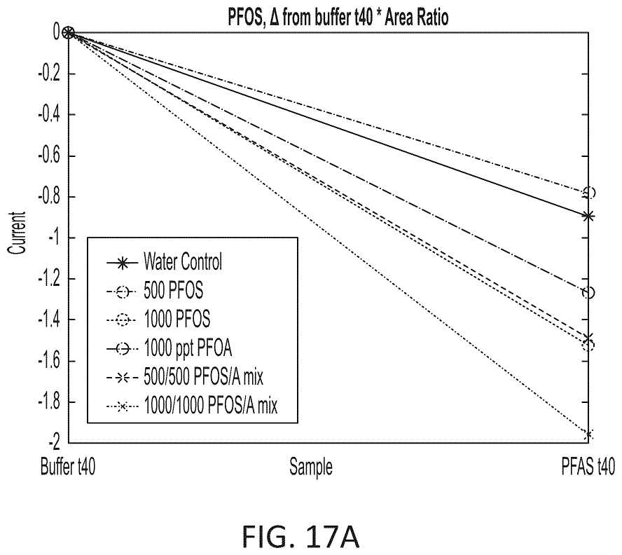

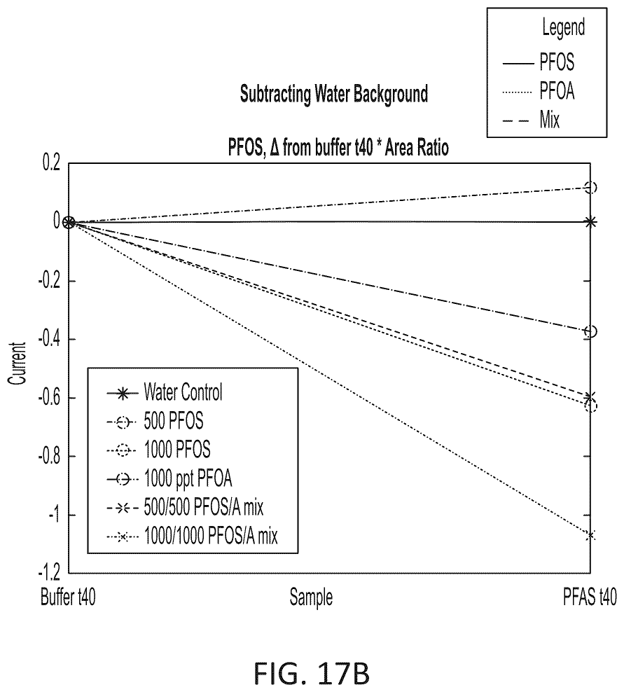

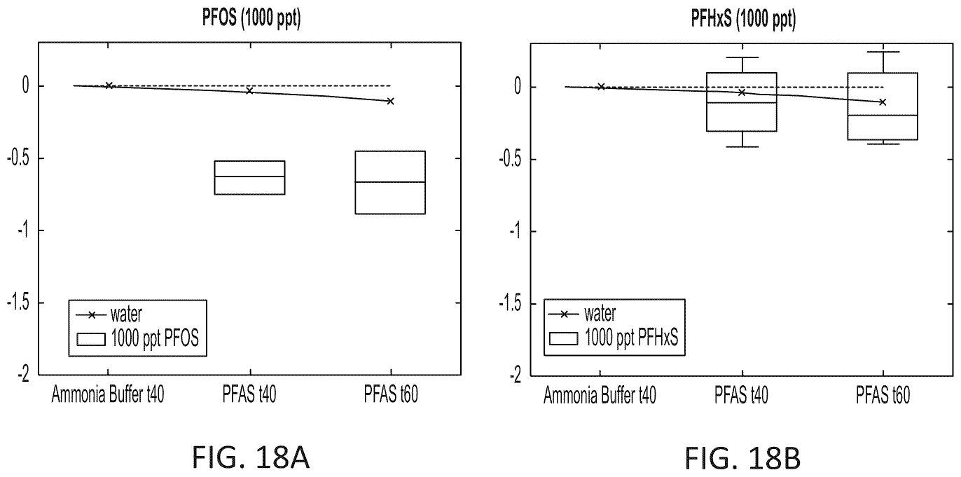

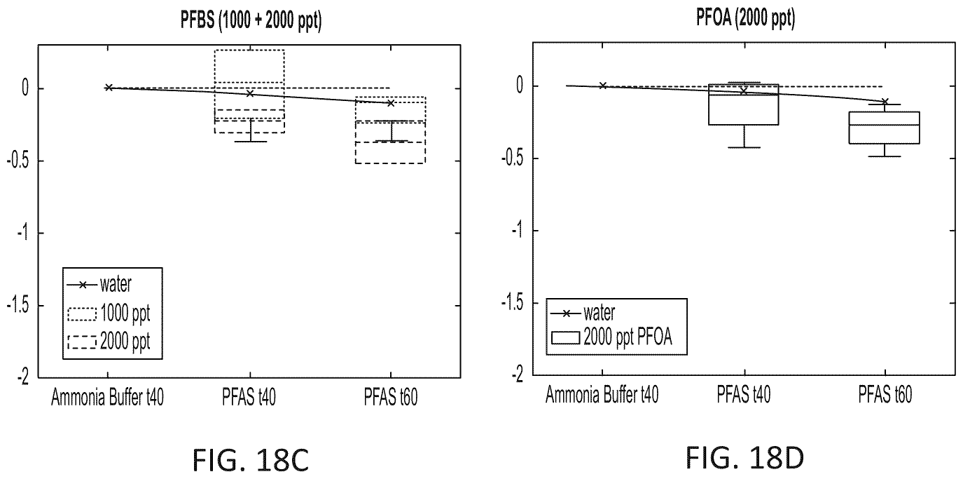

To extract the templates for the creation of more specific sensors (e.g., sensors used to produce the experimental results shown in FIGS. 18A-18D), a solution of approximately 0.20 M HCl was prepared and mixed in a 1:1 ratio with acetone to produce the extraction solution. For the creation of nonspecific sensors (e.g., sensors used to produce the experimental results shown in FIGS. 17A-17B), an extraction solution of 1:1 acetone:nanopure water was prepared and used to extract the templates. The WE was then placed into the extraction solution, without stirring, for 20 minutes.

### Extracted MIP Fc Characterization

Following extraction as described above (“Template Extraction”), the electrodes were characterized utilizing ferrocene carboxylic acid or another appropriate electron mediator utilizing CV and DPV as described above (“FcCOOH Characterization”).

After the analysis above, the WE was stored in the electrolyte solution consisting of pH 8.4 0.1 M ammonia buffer overnight.

### Testing in PFAS Solutions

The WEs modified with MIPs were then tested on spiked solutions containing various amounts of PFAS (e.g., 500/1000/2000 ppt). The spiked solutions were intended to represent the aqueous sample environment. The PFAS molecules of interest were dissolved in nanopure water to produce the aqueous sample environment solutions.

WEs modified with extracted MIPs were soaked in the desired parts-per-trillion PFAS solution for 20, 40, and 60 minutes total incubation time. After each soak in the aqueous sample environments, the WEs were moved to a separate sensing solution containing a solution of 1 mM FcCOOH+0.1 M ammonia at pH 8.4. The WE was characterized both immediately and after 5 min of incubation time, in the sensing solution utilizing with CV and DPV as described above (“FcCOOH Characterization”). The WEs were then placed back in the PFAS-contaminated aqueous sample environment for 20 minutes until the next measurement in the sensing solution. This process was repeated until data was collected for 20, 40, and 60 minutes of total incubation time in the aqueous sample environment was achieved and the response characterized.

### Exemplary Experimental Results

Testing of PFAS sensors may be conducted by incubating the prepared sensor in a solution containing amounts of PFAS to allow PFAS to bind to the affinity sites in the polymer layer of the sensor. The PFAS sensor may then be transferred to a sensing solution, as described above. A potentiostat may then be used to provide a potential difference between a working electrode and a reference electrode of the sensor, as described above with respect to method 900 shown in FIG. 9. The current between the working and reference electrodes can then be measured to indicate an amount of PFAS bound to the affinity sites. Control trials may be conducted by incubating the PFAS sensor in water or another solution lacking PFAS.

Several existing PFAS sensing systems were tested by initially incubating a PFAS sensor in water, and then gradually spiking the water with amounts of PFAS at regular intervals while current readings were taken on a potentiostat. The results of such trials showed decaying current flow between the electrodes over time, which was hypothesized to be a result of increased PFAS binding to the affinity sites due to the increasing concentration of PFAS and the spiking of the samples with PFAS during the trials. However, such trials may have provided inaccurate results, as many PFAS sensors produced by prior art methods failed to remain stable in aqueous solution for the length of time of the experimental trials.

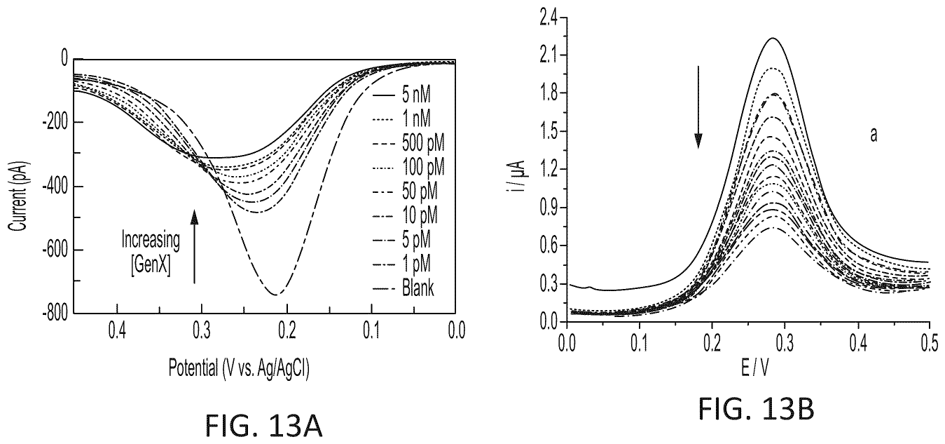

For example, FIGS. 13A-13B show the experimental results from two trials conducted on prior art PFAS sensors. In these trials, a PFAS sensor was dipped into a single container containing PFAS-contaminated water and an electron mediator. Current measurements were taken, and the sample was spiked with additional PFAS. This process was repeated a number of times in the same vial with increasing concentrations of PFAS. FIG. 13A illustrates the experimental results of a trial conducted in Glasscott, M. W., Vannoy, K. J., Kazemi, R., Verber, M. D., & Dick, J. E. μ-MIP: Molecularly Imprinted Polymer Modified Microelectrodes for the Ultrasensitive Quantification of GenX (HFPO-DA) in River Water. Environmental Science and Technology Letters, 7 (2020), pp. 489-495, incorporated by reference herein in its entirety. FIG. 13B illustrates the experimental results of a trial conducted in Karimian, N., Stortini, A. M., Moretto, L. M., Costantino, C., Bogialli, S., Ugo, P. Electrochemosensor for trace analysis of perfluorooctane sulfonate in water based on a molecularly imprinted poly o-phenylenediamine polymer. ACS Sensors, 3 (2018), pp. 1291-1298, incorporated by reference herein in its entirety. The results of each trail show that, over time, the measured current decreases over the course of the trial concurrently with the addition of PFAS, which was hypothesized to be the result of increasing PFAS concentration in the vials. However, neither study included experimental data conducted in water (i.e., water as a control trial without PFAS or PFAS spiking) over the same period of time.

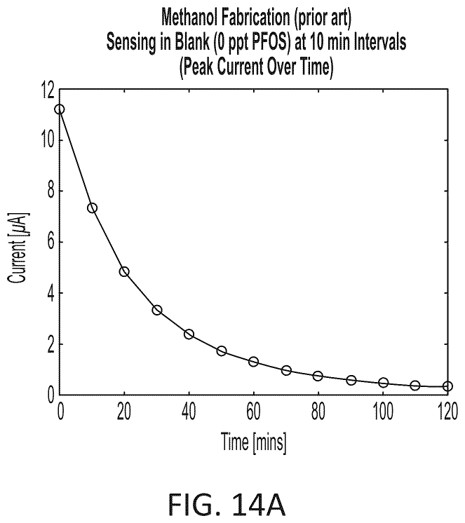

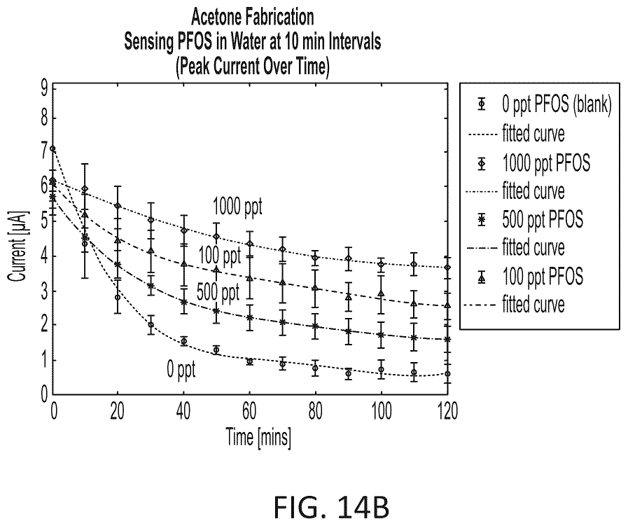

Additional data has indicated that PFAS sensors created by prior art methods produce experimental results showing similar current decay in water-only trials (i.e., control trials without PFAS spiking). For instance, FIG. 14A shows experimental results conducted on a PFAS sensor produced by prior art methods when the sensor is incubated in a water-only experimental solution. As shown in the graph of FIG. 14A, the peak current decays over time similarly to the trials shown in FIGS. 13A-13B, despite the lack of PFAS spiking of the experimental solution. Accordingly, it is hypothesized that the current decay shown in the experimental trials of FIGS. 13A-13B may be the result of changes in the mechanical or chemical composition of the PFAS sensor over time when exposed to the experimental solution, rather than the binding of the added PFAS to affinity sites on the sensor. For instance, the method of producing the prior art PFAS sensors (e.g., using methanol as a solvent during polymerization) may have resulted in a less robust polymer layer that tends to collapse, degrade, or otherwise change physical or chemical composition when exposed to an aqueous environment, resulting in the decay of current in all experimental trails irrespective of the presence of PFAS in the solution. After the collapse or degradation of the polymer layer of the sensor, it is hypothesized that the spiking with PFAS in these trails did not result in additional PFAS binding to the collapsed and/or degraded polymer layer, and that the current decay shown in the experimental results is not correlated with the concentration of PFAS in the solution.

FIG. 14B shows the experimental data from more recent experimental trials conducted on a PFAS sensor fabricated using an acetone solution (without acid) during electropolymerization of the polymer layer. The trials in FIG. 14B were initiated by incubating a PFAS sensor in prepared concentrations containing variable amount of PFOS (0 ppt PFOS, 100 ppt PFOS, 500 ppt PFOS, and 1000 ppt PFOS) and measuring the flow of current between the electrodes of the sensor over time using a potentiostat. In these experimental trials, the sensor was exposed to a single PFAS concentration for the duration of the experimental trial. For instance, the sensor was exposed to a solution containing either 0 ppt (parts-per-trillion) PFOS, 100 ppt PFOS, 500 ppt PFOS, or 1000 ppt PFOS, and current measurements were recorded over time while the sensor remained in the same solution with the same concentration of PFOS. In contrast to previous trails, the sensors were immediately exposed to solutions containing the indicated amount of PFAS without first exposing the sensors to water lacking PFAS. In other words, the concentration of PFAS in the experimental solutions remained constant, and no PFAS spiking occurred during the experimental period. As shown in the graph of FIG. 14B, the 0 ppt PFOS control trial experienced similar current decay as the PFAS spiking trials of FIGS. 13A-13B and the water-only control trial of FIG. 14A. These results indicate that the PFAS spiking in the trials of FIGS. 13A-13B did not result in significant changes in the measured current decay compared with control trials conducted in water alone. Sensors where the MIP is polymerized in acetone without acid shown in FIG. 14B show a similarly significant current decay in water only, without PFAS present. Interestingly, the experimental trials of FIG. 14B conducted in PFOS-containing solutions experienced significantly slower current decay than the current decay shown in the graphs of FIGS. 13A-13B and the water-alone control trials of FIGS. 14A and 14B. It is hypothesized that the reduced rates of current decay in the presence of PFAS seen in FIG. 14B are the result of the binding of PFOS to the affinity sites in the sensor, which may not have occurred in the PFAS-spiking trials where the sensors were initially exposed to water alone. Binding of PFAS to the affinity sites in the sensor may help maintain the structural integrity of the sites and reduce degradation of the affinity sites and polymer layer over time. This in turn may result in a more stable current over time. These results also demonstrate that the current response to PFAS may be non-monotonic with concentration, i.e., the 100 ppt PFOS current response falls between the 500 ppt and 1000 ppt current response. These results indicate that the sensor may not have a linear response function to PFAS concentration, as suggested in the prior art. Such results further indicate that the responses shown in FIGS. 13A and 13B may be primarily due to sensor interactions with the solution background or interferents and not solely due to binding with PFAS. Results shown in FIG. 14B indicate that fabrication via electropolymerization in methanol or acetone alone, without acid, does not result in a sensor with a linear response to PFAS concentration as proposed in prior art.

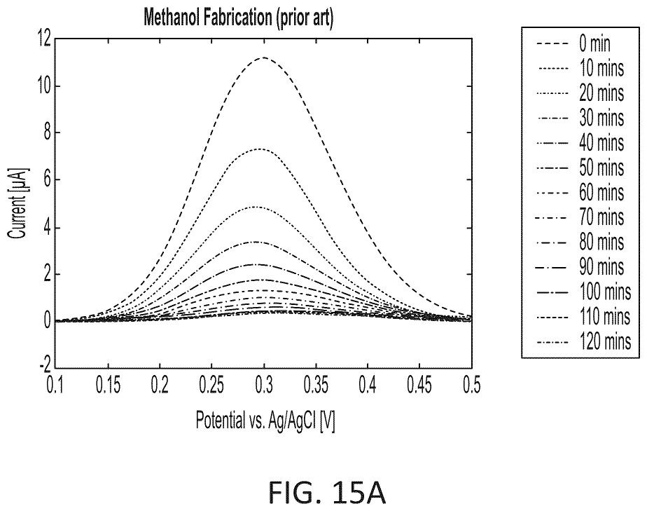

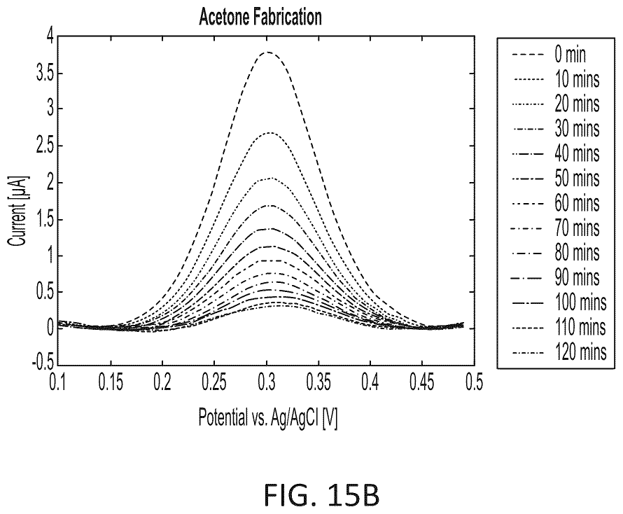

FIGS. 15A-15B show the results of experimental trails conducted by incubating a PFAS sensor in water alone. The current data was measured by differential pulse voltammetry and the sweeping of voltage across a range of potentials. The peak current detected during each voltage sweep is visible as the peak of the curve shown in the graphs. Each line represents a voltage sweep conducted at a particular timepoint during the experimental trial. The graph of FIG. 15A shows the current response of a PFAS sensor wherein polymerization of the polymer layer was performed in a methanol solution, while the graph of FIG. 15B shows the current response of a PFAS sensor wherein polymerization of the polymer layer was performed in an acetone solution. As seen in both graphs of FIGS. 15A-15B, trials conducted in water experienced similar decay in current over the experimental period for both acetone and methanol polymerization solutions. The current decay data measured in these trials also appears to replicate the current decay seen in the PFAS-spiking trials of FIGS. 13A-13B, providing further evidence that the current decay in the PFAS-spiking trials may be related to sensor degradation or some other phenomena in the water solution, rather than the sensor's binding with PFAS.

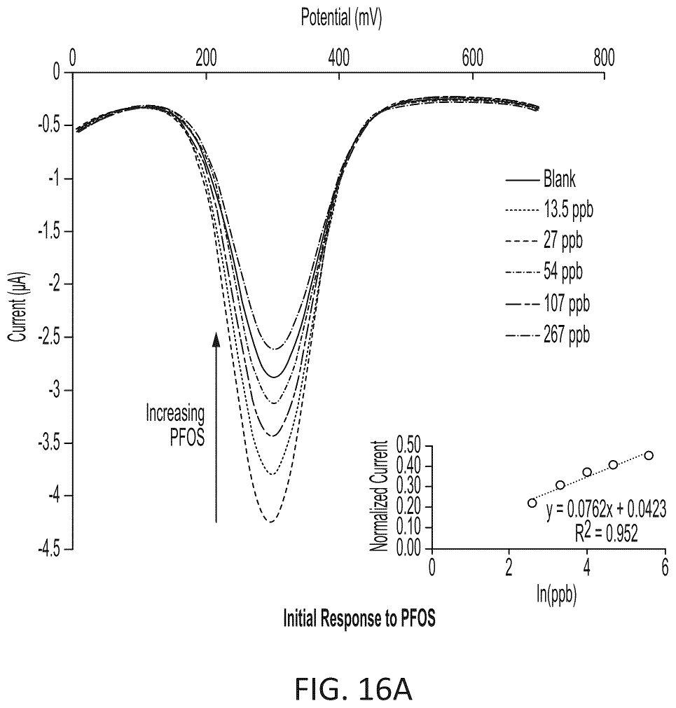

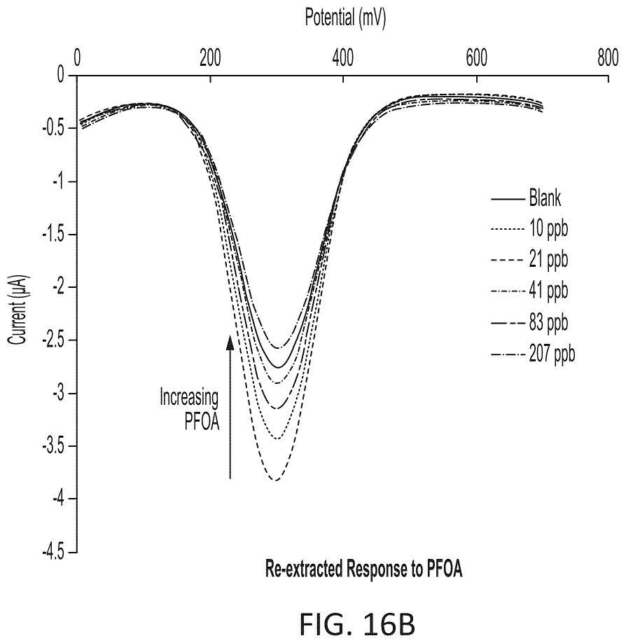

FIGS. 16A-16B show the results from trials conducted similarly to the trials of FIGS. 13A-13B, wherein a PFAS sensor fabricated by prior art methods was initially introduced to PFAS-free solution and the experimental solution was spiked with additional PFAS during the experimental period. The results in the graph were measured at various time points following the spiking of the solution with increasing amounts of PFAS. Similar to the trails of FIGS. 13A-13B, the current decayed throughout the experimental period. However, the current decay is hypothesized to be the result of the sensor degrading or collapsing in the experimental solution, as opposed to additional binding of the PFAS added during the experimental period. The graph of FIG. 16A shows the results of an experimental trial where the experimental solution was gradually spiked with additional concentrations of PFOS. The graph of FIG. 16B shows the results of an experimental trial where the experimental solution was gradually spiked with additional concentrations of PFOA. For these experimental trials, the sensor was fabricated in acetone with PFOS as the template molecule. As seen in FIGS. 16A-16B, both PFOS and PFOA spiking resulted in a similar current decay pattern (and a similar current decay pattern as the water-only trial of FIG. 14A). Accordingly, the particular species of PFAS did not have an effect on the current decay experienced by a prior art PFAS sensor in PFAS spiking trials initiated in a PFAS-free experimental solution. This provides further evidence that the current decay in the PFAS-spiking trials in FIGS. 13A-13B may be related to sensor degradation or another phenomena in the water solution, rather than the sensors' binding with PFAS.

Additional data was gathered after conducting experimental trials on PFAS sensors fabricated by the manufacturing methods disclosed herein. In the following experimental trials, the polymer layers of the PFAS sensors were manufactured by polymerizing monomers and PFAS templates in acidic solutions, which were hypothesized to produce polymer layers having more favorable mechanical properties in aqueous solutions (e.g., less likely to degrade or collapse prior to the binding of PFAS). Additionally, the PFAS sensors in the following trials were fabricated to be non-specific to a plurality of different PFAS. To create the nonspecific sensors, extraction of the template PFAS from the polymer layers was conducted by washing the PFAS sensors in an acetone and water solution, which was shown to create affinity sites in the polymer layer capable of binding with multiple PFAS species. Accordingly, the results of the following trials showed similar current decay behavior of the sensors following exposure to PFOS, PFOA, or a combination of both PFOS and PFOA.

FIGS. 17A-17B show experimental results conducted on a non-specific PFAS sensor fabricated by the manufacturing methods described herein. In these experimental trials, a PFAS sensor was incubated in an experimental solution containing various concentrations of PFOS and PFOA (e.g., 500 ppt PFOS, 1000 ppt PFOS, 500 ppt PFOS/500 ppt PFOA mix, and 1000 ppt PFOS/1000 ppt PFOA mix) and current readings were taken after 40 minutes. An additional control trial was conducted in an experimental solution lacking PFAS (water control). The left side of each graph provides current measurements in the buffer solution alone after 40 minutes of incubation, while the right side of each graph provides the change in current measurements taken after incubation in a PFAS-containing sample (or water control) after 40 minutes of incubation. Measurements were taken in the sensing solution with a potentiostat between electrodes of the PFAS sensor. FIG. 17A shows a comparison between “baseline” buffer current readings with current readings taken with the PFAS sensor in the presence of PFAS. FIG. 17B shows the results of the same experiment, where the water background signal is removed by subtracting the current due to the water from the remaining experimental results.

The lines of the graph with o's provide the results measured in solutions including amounts of PFOS alone or PFOA alone. The lines with x's provide the results measured in solutions including mixed amounts of both PFOS and PFOA. The black solid line with *'s provide the results measured in solutions with no PFAS present (uncontaminated water control). These results show a much smaller decrease in current from the water control itself when compared with the prior art that did not use acid during fabrication. This suggests the polymer layer is more mechanically and chemically robust and allows the sensor to distinguish the presence of PFAS at certain concentrations where the signal is stronger than the change in current in the water control. As shown in the results, the acid-fabricated sensor may be capable of non-specifically detecting PFOA, PFOS, and a combination of both PFOA and PFOS. For instance, the sensor may be operable to detect a total concentration of PFAS, represented by the sum of the concentration of each PFAS species present in the solution (e.g., the sum of the concentration of PFOA and PFOS in the experimental trails shown in FIGS. 17A-17B). For instance, the experimental results shown in FIGS. 17A-17B, the solid line corresponding to the 1000 ppt PFOS trial closely tracks the line corresponding to the mixed 500 ppt PFOA and 500 ppt PFOS trial. Additionally, after subtracting the background current decay in the water control, the sum of the current response to 1000 ppt PFOA (−0.4 μA) and 1000 ppt PFOS (−0.6 μA) is similar to the current response to a mix of 1000 ppt PFOA and 1000 ppt PFOS (−1.05 μA). Such results indicate that the current detected across a non-specific PFAS sensor may be used to detect and measure the concentration of multiple PFAS species with different molecular structures that are bound to the affinity sites in the polymer layer of the PFAS sensor. By characterizing the current response of the sensor to multiple different PFAS molecules at increasing concentrations, it is possible to determine a lower and upper bound on the total concentration of PFAS in mixed samples. This would be done by comparing the current response in an unknown, potentially mixed PFAS sample to the calibration curves for single PFAS species. The PFAS species with the strongest current response would be used to calculate the minimum PFAS concentration present, and the PFAS species with the weakest current response would be used to calculate the maximum PFAS concentration present. Accordingly, such non-specific sensors may be able to determine a cumulative amount of all PFAS species in an experimental solution containing multiple species of PFAS.

FIGS. 18A-18D show additional results from experimental trails conducted with a PFAS sensor manufactured according to the present disclosure. In particular, the PFAS sensor was fabricated using an acetone and acid electropolymerization solution and an acetone and acid extraction solution such that the sensor is specific to the template PFAS, in this case, PFOS. The sensor was then incubated in experimental solutions containing various amounts of PFAS species in nanopure water. FIG. 18A shows experimental results after incubation of the sensor in an experimental solution including 1000 ppt PFOS for 40 minutes and 60 minutes. FIG. 18B shows experimental results after incubation of the sensor in an experimental solution including 1000 ppt PFHxS. FIG. 18C shows experimental results after incubation of the sensor in an experimental solution including 1000 ppt PFBS and 2000 ppt PFBS. FIG. 18D shows experimental results after incubation of the sensor in an experimental solution including 2000 ppt PFOA.

It is hypothesized that extraction of PFAS templates using an acid and acetone solution produces PFAS sensors that are specific to particular PFAS species (i.e., PFAS species having molecular structures identical to or similar to a template PFAS species) and that do not degrade or collapse when exposed to water alone. The results demonstrate that the current measured between electrodes of the PFAS sensors experiences very little decay over time in the water-alone trials (represented by the solid black lines in the graphs). This provides evidence that PFAS sensors fabricated by the manufacturing methods described herein maintain relatively constant current and do not collapse or degrade in water alone. Further, the current response in the experimental trials including PFOS (represented by the box plots) shows significantly faster current decay that is distinguishable from the water alone trials. Experimental trials with PFHxS, PFBS, and PFOA showed minimal or indistinguishable current decay when compared to the water alone trials. The measured current decay may be the result of binding of the specific PFAS PFOS to the affinity sites, and the inability of other PFAS (e.g., PFHxS, PFBS, and PFOA) to bind to those same affinity sites. This suggests that when templated with a specific template PFAS, in this case PFOS, using an acid and acetone extraction solution to fabricate the sensors increases sensor specificity to that PFAS.

### CONCLUSION

Any one or more characteristics of any of the embodiments (including claims) described, shown, and/or referenced herein may be combined, in whole or in part, with any one or more characteristics of any one or more other embodiments (including claims) described, shown, and/or referenced herein.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method for quantifying amounts of per- and poly-fluoroalkyl substances (PFAS) using a PFAS detection system comprising:
a working electrode; and
a polymer layer disposed on a surface of the working electrode and comprising a plurality of affinity sites for detecting a plurality of PFAS molecules having different molecular structures, wherein each of the plurality of affinity sites were created using a template PFAS; and
one or more processors,
the method comprising:
detecting, at the working electrode, a plurality of PFAS molecules having different molecular structures that have bonded to one or more of the plurality of affinity sites; and
determining, by the one or more processors and based on the detected plurality of PFAS molecules that are bonded to the one or more affinity sites, a concentration of PFAS in an environment.

2. The method of claim 1, wherein the plurality of affinity sites comprise multiple affinity sites that were created using the same template PFAS.

3. The method of claim 2, wherein the multiple affinity sites that were created using the same template PFAS are configured to bind to one or more PFAS having molecular structures that are different from a molecular structure of the template PFAS.

4. The method of claim 3, wherein the one or more PFAS have molecular structures that are at least 60% identical to the molecular structure of the template PFAS.

5. The method of claim 3, wherein the multiple affinity sites that were created using the same template PFAS are configured to bind to at least two PFAS having molecular structures that are different from the molecular structure of the template PFAS.

6. The method of claim 1, wherein the PFAS detection system further comprises:
a second working electrode;
a second polymer layer disposed on a surface of the second working electrode and comprising a second plurality of affinity sites for detecting a second plurality of PFAS molecules, wherein each of the second plurality of affinity sites was creating using a second template PFAS different from the template PFAS.

7. The method of claim 1, wherein the determined concentration of PFAS in the environment is a total concentration of all PFAS having different molecular structures that can bind to the one or more affinity sites.

8. The method of claim 1, wherein detecting the plurality of PFAS molecules that are bonded to one or more of the plurality of affinity sites comprises:
generating, using a voltage source, a varying potential difference between the working electrode and a reference electrode; and
detecting, as the potential difference is varied, a current response of the working electrode.

9. The method of claim 8, wherein a potentiostat is used to generate the potential difference and to detect the current response.

10. The method of claim 8, wherein determining the concentration of PFAS in the environment comprises:
determining a difference between the detected current response of the working electrode to a current response of the working electrode in an uncontaminated environment; and
computing the concentration of PFAS based on the determined difference between the detected current response and the current response in the uncontaminated environment.

11. The method of claim 1, wherein the method comprises, prior to detecting the plurality PFAS molecules:
incubating the working electrode and the polymer layer in an aqueous sample environment containing PFAS molecules to allow one or more PFAS molecules to bind to one or more of the affinity sites; and
exposing the working electrode and the polymer layer to a sensing solution, wherein the detecting of the plurality of PFAS molecules occurs in the sensing solution.

12. The method of claim 1, wherein the method comprises, prior to detecting the plurality PFAS molecules, incubating the working electrode and the polymer layer in an electrolyte solution.

13. The method of claim 1, wherein the polymer layer is formed via molecular imprinting.

14. A system for quantifying amounts of per- and poly-fluoroalkyl substances (PFAS), the system comprising:
a working electrode;
a polymer layer disposed on a surface of the working electrode and comprising a plurality of affinity sites for detecting a plurality of PFAS molecules having different molecular structures, wherein each of the plurality affinity sites were created using a template PFAS; and
one or more processors;
wherein the working electrode is configured to detect a plurality of PFAS molecules having different molecular structures that have bonded to one or more of the plurality of affinity sites; and
wherein the one or more processors are configured to determine, based on the detected plurality of PFAS molecules that are bonded to the one or more affinity sites, a concentration of PFAS in an environment.

15. The system of claim 14, wherein the plurality of affinity sites comprise multiple affinity sites that were created using the same template PFAS.

16. The system of claim 15, wherein the multiple affinity sites that were created using the same template PFAS are configured to bind to one or more PFAS having molecular structures that are different from a molecular structure of the template PFAS.

17. The system of claim 16, wherein the one or more PFAS have molecular structures that are at least 60% identical to the molecular structure of the template PFAS.

18. The system of claim 16, wherein the multiple affinity sites that were created using the same template PFAS are configured to bind to at least two different PFAS having molecular structures that are different from the molecular structure of the template PFAS.

19. The system of claim 14, further comprising:
a second working electrode;
a second polymer layer disposed on a surface of the second working electrode and comprising a second plurality of affinity sites for detecting a second plurality of PFAS molecules, wherein each of the second plurality of affinity sites was creating using a second template PFAS different from the template PFAS.

20. The system of claim 14, wherein the determined concentration of PFAS in the environment is a total concentration of all PFAS having different molecular structures that can bind to the one or more affinity sites.

21. The system of claim 14, wherein detecting the plurality of PFAS molecules that are bonded to one or more of the plurality of affinity sites comprises:
generating a varying potential difference between the working electrode and a reference electrode; and
detecting, as the potential difference is varied, a current response of the working electrode.

22. The system of claim 21, wherein a potentiostat is used to generate the potential difference and to detect the current response.

23. The system of claim 21, wherein determining the concentration of PFAS in the environment comprises:
determining a difference between the detected current response of the working electrode to a current response of the working electrode in an uncontaminated environment; and
computing the concentration of PFAS based on the determined difference between the detected current response and the current response in the uncontaminated environment.

24. The system of claim 14, wherein the polymer layer is formed via molecular imprinting.

25. The system of claim 14, wherein the working electrode is operable to detect the plurality of PFAS molecules after exposing the polymer layer and the working electrode to an aqueous sample environment for at least 30 min.

26. A non-transitory computer readable storage medium storing instructions configured to be executed by a PFAS detection system comprising:
a working electrode;
a polymer layer disposed on a surface of the working electrode and comprising a plurality of affinity sites for detecting a plurality of PFAS molecules having different molecular structures, wherein each of the plurality affinity sites were created using a template PFAS; and
one or more processors,
wherein, when executed by the one or more processors, the instructions are configured to cause the PFAS detection system to:
detect, at the working electrode, a plurality PFAS molecules having different molecular structures that have bonded to one or more of the plurality of affinity sites; and
determine, by the one or more processors and based on the detected plurality of PFAS molecules that are bonded to the one or more affinity sites, a concentration of PFAS in an environment.

27. The non-transitory computer readable storage medium of claim 26, wherein the plurality of affinity sites comprise multiple affinity sites that were created using the same template PFAS.

28. The non-transitory computer readable storage medium of claim 27, wherein the multiple affinity sites that were created using the same template PFAS are configured to bind to one or more PFAS having molecular structures that are different from a molecular structure of the template PFAS.

29. The non-transitory computer readable storage medium of claim 28, wherein the one or more PFAS have molecular structures that are at least 60% identical to the molecular structure of the template PFAS.

30. The non-transitory computer readable storage medium of claim 28, wherein the multiple affinity sites that were created using the same template PFAS are configured to bind to at least two PFAS having molecular structures that are different from the molecular structure of the template PFAS.

31. The non-transitory computer readable storage medium of claim 26, wherein the PFAS detection system further comprises:
a second working electrode;
a second polymer layer disposed on a surface of the second working electrode and comprising a second plurality of affinity sites for detecting a second plurality of PFAS molecules, wherein each of the second plurality of affinity sites was creating using a second template PFAS different from the template PFAS.

32. The non-transitory computer readable storage medium of claim 26, wherein the determined concentration of PFAS in the environment is a total concentration of all PFAS having different molecular structures that can bind to the one or more affinity sites.

33. The non-transitory computer readable storage medium of claim 26, wherein detecting the plurality of PFAS molecules that are bonded to one or more of the plurality of affinity sites comprises:
generating, using a voltage source, a varying potential difference between the working electrode and a reference electrode; and
detecting, as the potential difference is varied, a current response of the working electrode.

34. The non-transitory computer readable storage medium of claim 33, wherein a potentiostat is used to generate the potential difference and to detect the current response.

35. The non-transitory computer readable storage medium of claim 33, wherein determining the concentration of PFAS in the environment comprises:
determining a difference between the detected current response of the working electrode to a current response of the working electrode in an uncontaminated environment; and
computing the concentration of PFAS based on the determined difference between the detected current response and the current response in the uncontaminated environment.

36. The non-transitory computer readable storage medium of claim 26, wherein the polymer layer is formed via molecular imprinting.

37. A method of manufacturing a PFAS sensor, the method comprising:
forming a polymer layer on a working electrode comprising: contacting the working electrode with a solution comprising a plurality of monomers and template PFAS, and an acid, and electropolymerizing the monomers to form the polymer layer and trap the template PFAS in the polymer layer;
extracting the template PFAS from the polymer layer to create a plurality of affinity sites in the polymer layer; and
incubating the working electrode and the polymer layer in an electrolyte solution.

38. The method of claim 37, wherein the acid is HCl.

39. The method of claim 37, wherein extracting the plurality of template PFAS comprises washing the polymer layer with an acetone solution.

40. The method of claim 39, wherein the acetone solution is a 1:1 acetone:water solution.

41. The method of claim 40, wherein the plurality of affinity sites are configured to bind to one or more PFAS having molecular structures that are different from a molecular structure of the template PFAS.

42. The method of claim 39, wherein the acetone solution is a 1:1: acetone:acid solution.

43. The method of claim 42, wherein the plurality of affinity sites are configured to bind to one or more PFAS having molecular structures that are the same as a molecular structure of the template PFAS.

44. The method of claim 37, wherein the electrolyte solution is an ammonia solution.

45. The method of claim 37, wherein the working electrode and the polymer layer are incubated in the electrolyte solution for at least 12 hours.

46. A PFAS sensor for quantifying amounts of per- and poly-fluoroalkyl substances (PFAS), the sensor comprising:
a working electrode; and
a polymer layer disposed on the working electrode and comprising a plurality of affinity sites for binding a plurality of PFAS molecules, wherein each of the plurality of affinity sites were created using a template PFAS; and
wherein the working electrode is operable to detect PFAS molecules bound to the affinity sites after exposing the working electrode and the polymer layer to an aqueous sample environment for at least 30 minutes.

47. The sensor of claim 46, wherein the polymer layer is stable in the aqueous sample environment for at least than 2 hours.

48. The sensor of claim 46, wherein the working electrode is operable to detect the PFAS molecules after exposing the working electrode and the polymer layer to the aqueous sample environment for at least 12 hours.

49. The sensor of claim 46, wherein the plurality of affinity sites are configured to bind to PFAS molecules having molecular structures that are different from a molecular structure of the template PFAS.

50. The system of claim 49, wherein the PFAS molecules have molecular structures that are at least 60% identical to the molecular structure of the template PFAS.

51. The sensor of claim 49, wherein the plurality of affinity sites are configured to bind to at least two PFAS molecules having molecular structures that are different from the molecular structure of the template PFAS.

52. The sensor of claim 46, further comprises:
a second working electrode;
a second polymer layer disposed on a surface of the second working electrode and comprising a second plurality of affinity sites for detecting a second plurality of PFAS molecules, wherein each of the second plurality of affinity sites was creating using a second template PFAS different from the template PFAS.

53. The sensor of claim 46, wherein the plurality of affinity sites are configured to bind to PFAS molecules having molecular structures that are the same as a molecular structure of the template PFAS.
